SEQUENCE LISTING (238 sequences)

SEQ ID NO: 1 - DNA, 120 bp, Homo sapiens
tggcgtgaacccgggaggcggagcttgcagtgagccgagatcgcgccactgcactccagcytgagagacagagtgatactccatctctaaatcaatcaatcaatcaatcaatcaatcaat

SEQ ID NO: 2 - DNA, 120 bp, Homo sapiens
agcttcagtggttttgctctctgttatgcccttcagaagctcctgttttcctcaatctgtycactggcctattaggacctacagtgcagggcctgaccagctatctgagtgaggtaaagg

SEQ ID NO: 3 - DNA, 120 bp, Homo sapiens
ctgaggcaggagaatggcgtgaacccgggaggcggagcttgcagtgagccgagatcgcgcyactgcactccagcttgagagacagagtgatactccatctctaaatcaatcaatcaatca

SEQ ID NO: 4 - DNA, 120 bp, Homo sapiens
ctctggctgggagcagtggctcacgcctgtaatcccagcactttgggaggctgaggtggayctcacgaggtcaggagattgagaccatcctggctaacacggtgaaaccccatctctact

SEQ ID NO: 5 - DNA, 120 bp, Homo sapiens
gggaagggccctgctgggtacgtaatcaaaaggtgcctgatgaaccccaccccacccagaygcaaatttacccacaaagggaggttctttgaaatggctcctttccaaaggctgagggac

SEQ ID NO: 6 - DNA, 120 bp, Homo sapiens
gattatttctgcatggcagtcattgacagtttctccttttaggctgagagaatctcctttyacacagcgattccctaggaaggtgtttaaaacagaaatagaataatggaaggaaaccga

SEQ ID NO: 7 - DNA, 120 bp, Homo sapiens
ataacattgctctttgtaggatcaagtgggatatatgtagaagagggcttgaagttgatcrtttggaaagacagccagtactgggatccataaaacttctattcaaaatgttaaatggat

SEQ ID NO: 8 - DNA, 120 bp, Homo sapiens
caaggtggtctctaacaggatgatgacatgtttactgaaaatgagagtttagaaatgaaastgtaggtagatcacagataacactgcatgggccttggagttgaatatattctggaaaat

SEQ ID NO: 9 - DNA, 120 bp, Homo sapiens
tccaaataatacttacagtcaagtggctgactcctcctatttcatggtcaaccaaattgcrtcattatcatcatcaacatcaccactatcattgttgtcatcattatctttattgagcaa

SEQ ID NO: 10 - DNA, 120 bp, Homo sapiens
tgcaggtatctgcagctacctccttcctgccacaacctctgctgatcagaaaggttatttmattttggaatttagtgctcattatatatgagaattgtacgtgataaataatatataatt

SEQ ID NO: 11 - DNA, 120 bp, Homo sapiens
catgggccttggagttgaatatattctggaaaatttaacatatttatcgtgttagatgcarccttacgttttacactggtgatcttttggtccatattgagaagtgtttgggtaaacatt

SEQ ID NO: 12 - DNA, 120 bp, Homo sapiens
tggtcatccattgtattcagatttctctctcttgctcaaactcatgatgacctacctgaartgtgtgcacccaggacagtccattctcttgtctagactgtaaattattcctactagact

SEQ ID NO: 13 - DNA, 120 bp, Homo sapiens
ggggtctaaatgaaaaactttgggagaagagcaacgtgctctgcactgaccagaagaaagrcattacttcagtattttctgtatatggcttgattatcccttatccaaaatgcatgctac

SEQ ID NO: 14 - DNA, 120 bp, Homo sapiens
gtttcgcccttgtgaggcacactgggcaatgctgccattcccattccacaggtgaggaaaytgagtctcagcgagactaaatgattttcctgaaaattatctgggaacactagagacact

SEQ ID NO: 15 - DNA, 120 bp, Homo sapiens
gaaccctagtaaatagaagtttcgcccttgtgaggcacactgggcaatgctgccattcccmttccacaggtgaggaaactgagtctcagcgagactaaatgattttcctgaaaattatct

SEQ ID NO: 16 - DNA, 120 bp, Homo sapiens
ggggggcttgaagcttctttcgcagagtttgcaaacagaaagaatgcataatggcaagaamgttaattgtccagggctgctccaggtagaaaggggcagagtaggcttgaactcgagcct

SEQ ID NO: 17 - DNA, 120 bp, Homo sapiens
caggtgaggaaactgagtctcagcgagactaaatgattttcctgaaaattatctgggaacrctagagacacttcaatttctagtcaggaaaggactggaagcgtcccagggctggggggc

SEQ ID NO: 18 - DNA, 120 bp, Homo sapiens
gaggcaattctgtttctgaataacccttgaaactcagaagggctctggcagtaccaccacygggcagaagagggcaacagaaccacattcagggagtacatccgtgcccaggactcctct

SEQ ID NO: 19 - DNA, 120 bp, Homo sapiens
tcacatttagtcagggagcatccgtctcatgcctggccgaggcaattctgtttctgaatamcccttgaaactcagaagggctctggcagtaccaccactgggcagaagagggcaacagaa

SEQ ID NO: 20 - DNA, 120 bp, Homo sapiens
caaggaatcaaaaatatctccttgccaggccgtgtggatcacatttagtcagggagcatcygtctcatgcctggccgaggcaattctgtttctgaataacccttgaaactcagaagggct

SEQ ID NO: 21 - DNA, 120 bp, Homo sapiens
tttcgattggcttttagaaacgctctttctgaaggaagtcttaacgtgtgactctgtcacytcagtctctaattatgctcaaactagtgatcaaggaatcaaaaatatctccttgccagg

SEQ ID NO: 22 - DNA, 120 bp, Homo sapiens
ttttcgattggcttttagaaacgctctttctgaaggaagtcttaacgtgtgactctgtcasttcagtctctaattatgctcaaactagtgatcaaggaatcaaaaatatctccttgccag

SEQ ID NO: 23 - DNA, 120 bp, Homo sapiens
tgacgacatgtgcccaaggtggtcggggcacagcctggttttatacattttagggagacakgagacatcaatcaatacatgtaagaagtacactggttccatccagaaagacggggacag

SEQ ID NO: 24 - DNA, 120 bp, Homo sapiens
agtgattggctcaggaaggggcatgggactgaataatggccaatgagctgagaggaaaaasacctgctggggcttccaggaaggtccctccttccttgccacctggtgtgtccagaggat

SEQ ID NO: 25 - DNA, 120 bp, Homo sapiens
caccaagagaacatgttctaggaggcaggaagaagcagctcgcagattcttaagtctcacrcctagaaacggacacagcatcacttctacctatgctgctggtcaaagcaggcacagagc

SEQ ID NO: 26 - DNA, 120 bp, Homo sapiens
gattcttaagtctcacgcctagaaacggacacagcatcacttctacctatgctgctggtcmaagcaggcacagagcccactggattgaagaaagaggcatagacccccacctttaaaggg

SEQ ID NO: 27 - DNA, 120 bp, Homo sapiens
cgtgcgaggtgctgcgctcacagtattactcatgcacctttctgataagaaaagtgaaaaygtgaagttgaaaactgaagacgcccagcaacttcctgaatccagccctccacgtcctgc

SEQ ID NO: 28 - DNA, 120 bp, Homo sapiens
actgggaacctccatagttaccacatgctgcgctgacttctctaacacgctttggccaatratgtttccctaatcacagcagctcctcatttagaatgtgttcttatttaggatgcgttc

SEQ ID NO: 29 - DNA, 120 bp, Homo sapiens
atgaaagtgctgttctcatcacttcctatccatggtccatgctgtccgtgtgacttaccayggtggacgttgaccttggccacctggctggctgttgggttctccactggaagtttactc

SEQ ID NO: 30 - DNA, 120 bp, Homo sapiens
acctatgctgctggtcaaagcaggcacagagcccactggattgaagaaagaggcatagacycccacctttaaagggctgagtgtcagagaacttgtggccaacattaatccaccacaact

SEQ ID NO: 31 - DNA, 120 bp, Homo sapiens
aagccctcaaatcccattcctaatctgatgagtctatggaccaatttgtggaggacagtakattaaatagatctgatttttgccatcaatgtaaggaggataaaaacttgcataccaatt

SEQ ID NO: 32 - DNA, 120 bp, Homo sapiens
ggaaaatatatatgatttgccactagatcaagaagtatggcagtgacaactcgtttgacayggttgcacgaaaagatcctgcaaaatcattttggagggaagcggcttagccttctctat

SEQ ID NO: 33 - DNA, 120 bp, Homo sapiens
cctcagtataaaagctctatcagtaccatgaacaatctcatcataatcacacttaatatcrtttcatataatcacaatcaaaactggaaacaattaaaacattttagcatatttttatag

SEQ ID NO: 34 - DNA, 120 bp, Homo sapiens
aggtggtttcatttgaggcctcatttgttaccattgaaatcaatgaaggtgactccccatrtcagagaaattccagatactaataagtagtccaggggagttttttggggagatgagggt

SEQ ID NO: 35 - DNA, 120 bp, Homo sapiens
ctgtctgccttgagaacttatgaaccatatggatccctggttcaacaaatacgaattctgytgctgggtccaattggagctgggaagtccagctttttcaactcagtgaggtctgttttc

SEQ ID NO: 36 - DNA, 120 bp, Homo sapiens
ggtccaattggagctgggaagtccagctttttcaactcagtgaggtctgttttccaagggyatgtaacgcatcaggctttggtgggcactaatacaactgggatatctgagaaggtaagc

SEQ ID NO: 37 - DNA, 120 bp, Homo sapiens
ggggtgtacaaattattgtattttaaagtcaatcagaatagtttattcttgtattataacmataacagttcactaattaaattaaatttaggaattgaattgttaagttaatttggtttt

SEQ ID NO: 38 - DNA, 120 bp, Homo sapiens
cataaattttagttacctcttccaagaggtggtttcatttgaggcctcatttgttaccatkgaaatcaatgaaggtgactccccatgtcagagaaattccagatactaataagtagtcca

SEQ ID NO: 39 - DNA, 120 bp, Homo sapiens
tccaagaggtggtttcatttgaggcctcatttgttaccattgaaatcaatgaaggtgactycccatgtcagagaaattccagatactaataagtagtccaggggagttttttggggagat

SEQ ID NO: 40 - DNA, 120 bp, Homo sapiens
acattgtagtacttgtaaataactagaaataacatgatttagtcataattgtgaaaaatartaataatttttcttggatttatgttctgtatctgtgaaaaaataaatttcttataaaac

SEQ ID NO: 41 - DNA, 120 bp, Homo sapiens
tgttatcaatggaagccttctcaaaaggaattgatttgcatatgcacaggcactccattcrgttgtcatcaaatgccctttgttcagagcttcatcatcggcaaaagtagatatgatgaa

SEQ ID NO: 42 - DNA, 120 bp, Homo sapiens
tgctgcttttaaaattatgaactatttcaaacttacagaaaagcacagagaacaatgaaayacctatgcactcacaagatttaattgtgttttttacattttatcagttcttcctcatca

SEQ ID NO: 43 - DNA, 120 bp, Homo sapiens
gatcctatttacagcattctatttattaatttttataaaaacctagtttattaaaaactasttacagtaatatttgattttttaaagacaattaggtcatttgtaaataataagttttcc

SEQ ID NO: 44 - DNA, 120 bp, Homo sapiens
gctaggattatttgcaggtttggttttttctcatttgtctgtggcattggagaatattctyggtttaaacagactaatgacttccttattgtccctgatattttgactatcttactattg

SEQ ID NO: 45 - DNA, 120 bp, Homo sapiens
ggaaaagagtaatgattctggaaagaaaggtgataagcctcagagtaagatcttcagggaytagcaagatgagctgggaaagaagagtgagagggagaagcatacccatcctgagagagt

SEQ ID NO: 46 - DNA, 120 bp, Homo sapiens
cctccactcctcagcgcccgcctccctgcatccctaggggcttccctactgccccgacctkcattccccggggtaaagcgagctctggagatcgcatagagaaactgtagtgtcctgggt

SEQ ID NO: 47 - DNA, 120 bp, Homo sapiens
aatgctcgggccaacgccactgcctgtcgctgaccccctgacagctggctcccagcctcgyctacctctgcagagcaaagggccaagatgagaacggtatagccacatgtgtgcacgcat

SEQ ID NO: 48 - DNA, 120 bp, Homo sapiens
aactgtgagaaataaatttctgttcttcataaattaccctatctcgtgtatcttgttacamtaacacaaatggactaagacagagagcataaggcttgggggaagaagggtacacttctt

SEQ ID NO: 49 - DNA, 120 bp, Homo sapiens
tcagatcatcttcttctgtgagggctgcagcttccatgtagttgggagatacaggaattaytattcctgttttatgaataaaggacatttgtgggagagaaaggaatcaggccagagttc

SEQ ID NO: 50 - DNA, 120 bp, Homo sapiens
cctgatttaagcaaagtataataaacacactcatacacatatactacatggataccacaartggaaatttgacaattgactatttgataaattttaagaactactgttaattttttggtg

SEQ ID NO: 51 - DNA, 120 bp, Homo sapiens
tcatagtcaattactctgtgttgggtctacaccacatctgcacatactatgagcccttccrttggagataattttcacttgcggagctgcttcacttctacctgtaggagcctcatctcc

SEQ ID NO: 52 - DNA, 120 bp, Homo sapiens
attggctcatactactgtgggagctggcacggtcgaaatctgcaggtaggctggagacccrggaagagctgatgttgcggcttgagtctgaaggtggtccagaggcagaattccctcttc

SEQ ID NO: 53 - DNA, 120 bp, Homo sapiens
gtgaagtgacaacagcttcagctcgtcatcgatgttatacagaaacacaaaaggaatcccyggcctgtgatgaaggagaggccgttgctgtgtgttcaggacacctcagagcaggcacat

SEQ ID NO: 54 - DNA, 120 bp, Homo sapiens
cagaaacacaaaaggaatccccggcctgtgatgaaggagaggccgttgctgtgtgttcagracacctcagagcaggcacataaagtgctggagggtgacacagcctgtctggatgtcctc

SEQ ID NO: 55 - DNA, 120 bp, Homo sapiens
ggagggtgtgccatccaggaggcgacacccccatccagcacacgggcccttccacccgctrtcggtcctgctaaaggtacgaggctaaaaccggcctctccagaaaagaacgctcagtgt

SEQ ID NO: 56 - DNA, 120 bp, Homo sapiens
aaaactgtcatgatgacaaaatgcaagcatgacgtaaaatgcctaggtcagtgcctggcayacagcacatgctgggtaagcgcctgatattctgatgctgctctcctccctacagactct

SEQ ID NO: 57 - DNA, 120 bp, Homo sapiens
cttccctctagccaacaggtgctttttcactgccagcatttctcagcctccaggacaggcygagtcttgctcatggctcccctccctcctccaggcccacaagctccatgttggcagtgg

SEQ ID NO: 58 - DNA, 120 bp, Homo sapiens
cgttgctgtgtgttcaggacacctcagagcaggcacataaagtgctggagggtgacacagyctgtctggatgtcctcgggagggtgtgccatccaggaggcgacacccccatccagcaca

SEQ ID NO: 59 - DNA, 120 bp, Homo sapiens
aatgtggggttctcaactggcctgggaggcagctctgggcacgcccatttcctgagcatgmgactgctctctgcctcaatttcctctcctgtgaaatggagatgctgacagtaaatactg

SEQ ID NO: 60 - DNA, 120 bp, Homo sapiens
cgtaaaatgcctaggtcagtgcctggcacacagcacatgctgggtaagcgcctgatattcygatgctgctctcctccctacagactctcttaatcaccagcgtccaaaaggggagaaaaa

SEQ ID NO: 61 - DNA, 120 bp, Homo sapiens
gacaatactgagatcatcctcaccgagaagaaagtggactcggtataaatcagatttctgragcagccgccgcagcttggcctgcgtctcggtggcctctgcacccttgccactggcgga

SEQ ID NO: 62 - DNA, 120 bp, Homo sapiens
agggcccagtgtggcccaggtctgggtgcttcctcctcccaaagatcaagtccttcagggmaaccacctaatcctgctccagaaaacagcagtgtcagacttctgaggggtcgaggagcg

SEQ ID NO: 63 - DNA, 120 bp, Homo sapiens
tgaagagaatacccagctgagcctggaaaccaaggcagaaaagcaacacccaggataacaygccatcagagtctgcgcaaaggcaccatcacagctctgctgaaaccagcattttcctgg

SEQ ID NO: 64 - DNA, 120 bp, Homo sapiens
aaagcaacacccaggataacacgccatcagagtctgcgcaaaggcaccatcacagctctgytgaaaccagcattttcctggcactaaattacaaacagatttgttgaatggtccttgaag

SEQ ID NO: 65 - DNA, 120 bp, Homo sapiens
agaaatattcatgtggccatttctgtggggactctcgtagaatttcagagcctaacattgkaatgcaacaaacagttcctttcccatctcctctccggaacctccttgtcctggctacaa

SEQ ID NO: 66 - DNA, 120 bp, Homo sapiens
gaatggcgaggaaggacccaggatccatgctgcctgcgatgttcagagcatcctccttcayccaagctctgatcagctgtcctcctctactggcttccacctctggctgcccttccttcc

SEQ ID NO: 67 - DNA, 120 bp, Homo sapiens
cacccaggctttcttagtcagagatgcagaaatgctgcatgttccatctcctgctggtgaygtccaacccacatgaacacaccaacatccgacactcctgcaataaaggggccagtttat

SEQ ID NO: 68 - DNA, 120 bp, Homo sapiens
tctctgatcccacgccccactccgtcaccgtccagcatggctggtgtcctcgtcttgccaytgtctatatgaaatggctcctggacatgtttcttcactctgttcctacagatgccaaca

SEQ ID NO: 69 - DNA, 120 bp, Homo sapiens
cagctatagtaagaattttttgagaaagatgacaaccacaaccaaatgagtctgcaaataycacagcaggacacacaccatgtaaaccctggagctgagggaagatgaacaggcacacgg

SEQ ID NO: 70 - DNA, 120 bp, Homo sapiens
agagtagctgaattatttaccttcttcaagaagcactgtcataggacagggtttaagactwtaaacctctggtttaaagtctggtggttactatgctgaagatagaatctgtatataggt

SEQ ID NO: 71 - DNA, 120 bp, Homo sapiens
gaatttaacaacgtaataatccctaaaggaaaacacactgcctccttctcatctgtcgctwgtccatatgaaaagtagcaacgtctggtggacaagggcagcctcagaatgggctctggg

SEQ ID NO: 72 - DNA, 120 bp, Homo sapiens
cagattcccacacactggacttcctctgactggctgatggcagggatcctggctttaggcrtgagtatctttctttctttgcatcttcctacaatttacagtttttgtataaatacaatg

SEQ ID NO: 73 - DNA, 120 bp, Homo sapiens
ctcaaggttacctggctacaaggtgatgaaacagggctttgaccctagtttttgttcgtgwctccaaggccatattcttattgattgaggggaacaagtatgatttcagatcctctccta

SEQ ID NO: 74 - DNA, 120 bp, Homo sapiens
cccctaccctagtgtgactcatggccacataaatgccccatccccagtggtgcttcagctstgacccagtggaaccagtgtcaccggctcagcctccaggtaggggactggcttcccagg

SEQ ID NO: 75 - DNA, 22 bp, Artificial Sequence
  Primer
gaggtgaaaatgggaacaaagg

SEQ ID NO: 76 - DNA, 22 bp, Artificial Sequence
  Primer
aaaactacaaccaagcctgtcc

SEQ ID NO: 77 - DNA, 23 bp, Artificial Sequence
  Primer
taagccgttttctgagagaggtg

SEQ ID NO: 78 - DNA, 22 bp, Artificial Sequence
  Primer
ctctttgctcagtctgggattt

SEQ ID NO: 79 - DNA, 23 bp, Artificial Sequence
  Primer
ggagagaatatgggagtctagga

SEQ ID NO: 80 - DNA, 23 bp, Artificial Sequence
  Primer
gcatagttagccttttgggtctc

SEQ ID NO: 81 - DNA, 23 bp, Artificial Sequence
  Primer
ttcacatttttatctggacactt

SEQ ID NO: 82 - DNA, 23 bp, Artificial Sequence
  Primer
acactttacagtcaaaggcatac

SEQ ID NO: 83 - DNA, 24 bp, Artificial Sequence
  Primer
catgtttactgaaaatgagagttt

SEQ ID NO: 84 - DNA, 23 bp, Artificial Sequence
  Primer
atgataccaaaattgaagacaag

SEQ ID NO: 85 - DNA, 21 bp, Artificial Sequence
  Primer
gactcctcctatttcatggtc

SEQ ID NO: 86 - DNA, 23 bp, Artificial Sequence
  Primer
gccccatacttgatattagatgt

SEQ ID NO: 87 - DNA, 22 bp, Artificial Sequence
  Primer
gagaatccaaatgcagatagag

SEQ ID NO: 88 - DNA, 22 bp, Artificial Sequence
  Primer
atgtttttcagggatagacaaa

SEQ ID NO: 89 - DNA, 24 bp, Artificial Sequence
  Primer
catgtttactgaaaatgagagttt

SEQ ID NO: 90 - DNA, 23 bp, Artificial Sequence
  Primer
atgataccaaaattgaagacaag

SEQ ID NO: 91 - DNA, 23 bp, Artificial Sequence
  Primer
tccattgtattcagatttctctc

SEQ ID NO: 92 - DNA, 23 bp, Artificial Sequence
  Primer
taagcacttttaagcattttgag

SEQ ID NO: 93 - DNA, 22 bp, Artificial Sequence
  Primer
aaggggtctaaatgaaaaactt

SEQ ID NO: 94 - DNA, 23 bp, Artificial Sequence
  Primer
cacatgatattcaaaggaaatgt

SEQ ID NO: 95 - DNA, 23 bp, Artificial Sequence
  Primer
aagaacaaggaccaagaggacag

SEQ ID NO: 96 - DNA, 23 bp, Artificial Sequence
  Primer
cttgtagagctcaaaggcatacg

SEQ ID NO: 97 - DNA, 23 bp, Artificial Sequence
  Primer
aggtttattcgacaaagttaagg

SEQ ID NO: 98 - DNA, 23 bp, Artificial Sequence
  Primer
ggttgtctctaaactcaaaagaa

SEQ ID NO: 99 - DNA, 20 bp, Artificial Sequence
  Primer
actgaataatggccaatgag

SEQ ID NO: 100 - DNA, 20 bp, Artificial Sequence
  Primer
aggttccttgactccaactc

SEQ ID NO: 101 - DNA, 21 bp, Artificial Sequence
  Primer
agcagcataaatggacttctt

SEQ ID NO: 102 - DNA, 21 bp, Artificial Sequence
  Primer
cagttgtggtggattaatgtt

SEQ ID NO: 103 - DNA, 20 bp, Artificial Sequence
  Primer
tccacacctcaaggtagttc

SEQ ID NO: 104 - DNA, 20 bp, Artificial Sequence
  Primer
gtgaggtgagatcctgaaac

SEQ ID NO: 105 - DNA, 21 bp, Artificial Sequence
  Primer
gactgggaacctccatagtta

SEQ ID NO: 106 - DNA, 23 bp, Artificial Sequence
  Primer
gtatcctacgggacagagtttat

SEQ ID NO: 107 - DNA, 22 bp, Artificial Sequence
  Primer
gcttttctgatgtttttctcat

SEQ ID NO: 108 - DNA, 24 bp, Artificial Sequence
  Primer
tctttaatggatttagtgagtctg

SEQ ID NO: 109 - DNA, 23 bp, Artificial Sequence
  Primer
ggacacagcattacttctaccta

SEQ ID NO: 110 - DNA, 22 bp, Artificial Sequence
  Primer
actccacagaaaagatcaatgt

SEQ ID NO: 111 - DNA, 23 bp, Artificial Sequence
  Primer
atgctaaggtacccacaagatgg

SEQ ID NO: 112 - DNA, 21 bp, Artificial Sequence
  Primer
cttcttggccatgggaatttg

SEQ ID NO: 113 - DNA, 23 bp, Artificial Sequence
  Primer
tttgccactagatcaagaagtat

SEQ ID NO: 114 - DNA, 23 bp, Artificial Sequence
  Primer
catactttccttcctggtaactc

SEQ ID NO: 115 - DNA, 23 bp, Artificial Sequence
  Primer
atcagtaccatgaacaatctcat

SEQ ID NO: 116 - DNA, 25 bp, Artificial Sequence
  Primer
aaaccaaattaacttaacaattcaa

SEQ ID NO: 117 - DNA, 22 bp, Artificial Sequence
  Primer
tagagccctttggtctttagtc

SEQ ID NO: 118 - DNA, 21 bp, Artificial Sequence
  Primer
ctgggcaacagaaacttcatc

SEQ ID NO: 119 - DNA, 23 bp, Artificial Sequence
  Primer
cctaacctcagtcaattgttaaa

SEQ ID NO: 120 - DNA, 23 bp, Artificial Sequence
  Primer
aaatgtgcttaccttctcagata

SEQ ID NO: 121 - DNA, 22 bp, Artificial Sequence
  Primer
aaacaggctcaggaagagctta

SEQ ID NO: 122 - DNA, 23 bp, Artificial Sequence
  Primer
ctgccttcagacttcctcttaca

SEQ ID NO: 123 - DNA, 23 bp, Artificial Sequence
  Primer
agtatctaagaccaaagggatgt

SEQ ID NO: 124 - DNA, 23 bp, Artificial Sequence
  Primer
aaagcagtgaatatcagaagatg

SEQ ID NO: 125 - DNA, 22 bp, Artificial Sequence
  Primer
gcattaccttctacaattggtc

SEQ ID NO: 126 - DNA, 24 bp, Artificial Sequence
  Primer
gtgaagtactaatgtctgcaaaac

SEQ ID NO: 127 - DNA, 23 bp, Artificial Sequence
  Primer
aaaggatacaccatttctattca

SEQ ID NO: 128 - DNA, 24 bp, Artificial Sequence
  Primer
tgaggaagaactgataaaatgtaa

SEQ ID NO: 129 - DNA, 24 bp, Artificial Sequence
  Primer
gcataggttctacatgttttatgt

SEQ ID NO: 130 - DNA, 23 bp, Artificial Sequence
  Primer
aatcagaaaagtgaaggaaaact

SEQ ID NO: 131 - DNA, 23 bp, Artificial Sequence
  Primer
ctcttttcctacctgaactcttc

SEQ ID NO: 132 - DNA, 23 bp, Artificial Sequence
  Primer
taaacccagatgaaaatagatgt

SEQ ID NO: 133 - DNA, 23 bp, Artificial Sequence
  Primer
agtgaagggaaaagagtaatgat

SEQ ID NO: 134 - DNA, 23 bp, Artificial Sequence
  Primer
ttaagtctcagatggaatgtctc

SEQ ID NO: 135 - DNA, 21 bp, Artificial Sequence
  Primer
ctaacttgcacttcctcctct

SEQ ID NO: 136 - DNA, 23 bp, Artificial Sequence
  Primer
aggacactacagtttctctatgc

SEQ ID NO: 137 - DNA, 23 bp, Artificial Sequence
  Primer
catcatccaggataagtacacac

SEQ ID NO: 138 - DNA, 21 bp, Artificial Sequence
  Primer
ttctgttcatcttcccagaat

SEQ ID NO: 139 - DNA, 22 bp, Artificial Sequence
  Primer
ggtatcatggtaaccacaagtt

SEQ ID NO: 140 - DNA, 23 bp, Artificial Sequence
  Primer
aatcgtcctgtctttattctttt

SEQ ID NO: 141 - DNA, 23 bp, Artificial Sequence
  Primer
taggtttgactgttgcattattt

SEQ ID NO: 142 - DNA, 23 bp, Artificial Sequence
  Primer
aatttcacagagggtaaaatagg

SEQ ID NO: 143 - DNA, 23 bp, Artificial Sequence
  Primer
tattctgaagcaaatccaagata

SEQ ID NO: 144 - DNA, 22 bp, Artificial Sequence
  Primer
agtcacaatatgtggaccattt

SEQ ID NO: 145 - DNA, 23 bp, Artificial Sequence
  Primer
caattactctgtgttgggtctac

SEQ ID NO: 146 - DNA, 23 bp, Artificial Sequence
  Primer
tcatctttccactttctctattg

SEQ ID NO: 147 - DNA, 23 bp, Artificial Sequence
  Primer
atagattttgggtaaagcagatt

SEQ ID NO: 148 - DNA, 23 bp, Artificial Sequence
  Primer
ttggcagagagagatttatttta

SEQ ID NO: 149 - DNA, 23 bp, Artificial Sequence
  Primer
tcgatgttatacagaaacacaaa

SEQ ID NO: 150 - DNA, 20 bp, Artificial Sequence
  Primer
acacactgagcgttcttttc

SEQ ID NO: 151 - DNA, 20 bp, Artificial Sequence
  Primer
agaatgtggggttctcaact

SEQ ID NO: 152 - DNA, 23 bp, Artificial Sequence
  Primer
attaagagagtctgtagggagga

SEQ ID NO: 153 - DNA, 20 bp, Artificial Sequence
  Primer
atactgtcctgagcctgcta

SEQ ID NO: 154 - DNA, 22 bp, Artificial Sequence
  Primer
aagtctgacactgctgttttct

SEQ ID NO: 155 - DNA, 23 bp, Artificial Sequence
  Primer
tcgatgttatacagaaacacaaa

SEQ ID NO: 156 - DNA, 20 bp, Artificial Sequence
  Primer
acacactgagcgttcttttc

SEQ ID NO: 157 - DNA, 23 bp, Artificial Sequence
  Primer
aagggagacaatactgagatcat

SEQ ID NO: 158 - DNA, 20 bp, Artificial Sequence
  Primer
ctttgactttcccacttgaa

SEQ ID NO: 159 - DNA, 20 bp, Artificial Sequence
  Primer
atactgtcctgagcctgcta

SEQ ID NO: 160 - DNA, 22 bp, Artificial Sequence
  Primer
aagtctgacactgctgttttct

SEQ ID NO: 161 - DNA, 23 bp, Artificial Sequence
  Primer
ggtgtagggtagcttgagatcag

SEQ ID NO: 162 - DNA, 22 bp, Artificial Sequence
  Primer
aaaagaacagaagggacgaagg

SEQ ID NO: 163 - DNA, 22 bp, Artificial Sequence
  Primer
aactcacagtaccacctttcag

SEQ ID NO: 164 - DNA, 23 bp, Artificial Sequence
  Primer
gatgtaacaagaacaaagtggtc

SEQ ID NO: 165 - DNA, 23 bp, Artificial Sequence
  Primer
gttcttgttacatccagcaatag

SEQ ID NO: 166 - DNA, 23 bp, Artificial Sequence
  Primer
ggtgtcaaagtcagagagagtaa

SEQ ID NO: 167 - DNA, 24 bp, Artificial Sequence
  Primer
tcagactagaatatttttcacagc

SEQ ID NO: 168 - DNA, 20 bp, Artificial Sequence
  Primer
tgtccctgaattagccatag

SEQ ID NO: 169 - DNA, 23 bp, Artificial Sequence
  Primer
gctgaattatttaccttcttcaa

SEQ ID NO: 170 - DNA, 23 bp, Artificial Sequence
  Primer
aatctttatgatgcactgtgtct

SEQ ID NO: 171 - DNA, 23 bp, Artificial Sequence
  Primer
ggatcgaatttaacaacgtaata

SEQ ID NO: 172 - DNA, 24 bp, Artificial Sequence
  Primer
caaaaactgtaaattgtaggaaga

SEQ ID NO: 173 - DNA, 23 bp, Artificial Sequence
  Primer
tggagatacagagatcttaggtg

SEQ ID NO: 174 - DNA, 21 bp, Artificial Sequence
  Primer
gaagcaatagcaaagactcct

SEQ ID NO: 175 - DNA, 21 bp, Artificial Sequence
  Primer
caacaatcttcctgtgacttg

SEQ ID NO: 176 - DNA, 20 bp, Artificial Sequence
  Primer
gctcttgtaccttcccaatc

SEQ ID NO: 177 - DNA, 23 bp, Artificial Sequence
  Primer
ccagtactggctgtctttccaaa

SEQ ID NO: 178 - DNA, 26 bp, Artificial Sequence
  Primer
gcagtgttatctgtgatctacctaca

SEQ ID NO: 179 - DNA, 25 bp, Artificial Sequence
  Primer
gtggtgatgttgatgatgataatga

SEQ ID NO: 180 - DNA, 25 bp, Artificial Sequence
  Primer
cctctgctgatcagaaaggttattt

SEQ ID NO: 181 - DNA, 27 bp, Artificial Sequence
  Primer
aaaagatcaccagtgtaaaacgtaagg

SEQ ID NO: 182 - DNA, 26 bp, Artificial Sequence
  Primer
ctcaaactcatgatgacctacctgaa

SEQ ID NO: 183 - DNA, 18 bp, Artificial Sequence
  Primer
ctgtcctgggtgcacaca

SEQ ID NO: 184 - DNA, 23 bp, Artificial Sequence
  Primer
gctctgcactgaccagaagaaag

SEQ ID NO: 185 - DNA, 32 bp, Artificial Sequence
  Primer
tcaagccatatacagaaaatactgaagtaatg

SEQ ID NO: 186 - DNA, 28 bp, Artificial Sequence
  Primer
gcctggttttatacattttagggagaca

SEQ ID NO: 187 - DNA, 17 bp, Artificial Sequence
  Primer
tggaagccccagcaggt

SEQ ID NO: 188 - DNA, 22 bp, Artificial Sequence
  Primer
tgatgctgtgtccgtttctagg

SEQ ID NO: 189 - DNA, 24 bp, Artificial Sequence
  Primer
ggcgtcttcagttttcaacttcac

SEQ ID NO: 190 - DNA, 23 bp, Artificial Sequence
  Primer
agctgctgtgattagggaaacat

SEQ ID NO: 191 - DNA, 20 bp, Artificial Sequence
  Primer
gctgtccgtgtgacttacca

SEQ ID NO: 192 - DNA, 26 bp, Artificial Sequence
  Primer
actggattgaagaaagaggcatagac

SEQ ID NO: 193 - DNA, 22 bp, Artificial Sequence
  Primer
ggcagtgacaactcgtttgaca

SEQ ID NO: 194 - DNA, 21 bp, Artificial Sequence
  Primer
gcaggatcttttcgtgcaacc

SEQ ID NO: 195 - DNA, 34 bp, Artificial Sequence
  Primer
aattgtttccagttttgattgtgattatatgaaa

SEQ ID NO: 196 - DNA, 24 bp, Artificial Sequence
  Primer
gaacaaagggcatttgatgacaac

SEQ ID NO: 197 - DNA, 26 bp, Artificial Sequence
  Primer
acagaaaagcacagagaacaatgaaa

SEQ ID NO: 198 - DNA, 39 bp, Artificial Sequence
  Primer
atgacctaattgtctttaaaaaatcaaatattactgtaa

SEQ ID NO: 199 - DNA, 25 bp, Artificial Sequence
  Primer
tgtctgtggcattggagaatattct

SEQ ID NO: 200 - DNA, 31 bp, Artificial Sequence
  Primer
acaataaggaagtcattagtctgtttaaacc

SEQ ID NO: 201 - DNA, 24 bp, Artificial Sequence
  Primer
gcctcagagtaagatcttcaggga

SEQ ID NO: 202 - DNA, 17 bp, Artificial Sequence
  Primer
ccctactgccccgacct

SEQ ID NO: 203 - DNA, 16 bp, Artificial Sequence
  Primer
ctggctcccagcctcg

SEQ ID NO: 204 - DNA, 29 bp, Artificial Sequence
  Primer
aattaccctatctcgtgtatcttgttaca

SEQ ID NO: 205 - DNA, 26 bp, Artificial Sequence
  Primer
catgtagttgggagatacaggaatta

SEQ ID NO: 206 - DNA, 38 bp, Artificial Sequence
  Primer
tcttaaaatttatcaaatagtcaattgtcaaatttcca

SEQ ID NO: 207 - DNA, 25 bp, Artificial Sequence
  Primer
ctccgcaagtgaaaattatctccaa

SEQ ID NO: 208 - DNA, 20 bp, Artificial Sequence
  Primer
gccgcaacatcagctcttcc

SEQ ID NO: 209 - DNA, 28 bp, Artificial Sequence
  Primer
tgttatacagaaacacaaaaggaatccc

SEQ ID NO: 210 - DNA, 20 bp, Artificial Sequence
  Primer
cctctccttcatcacaggcc

SEQ ID NO: 211 - DNA, 23 bp, Artificial Sequence
  Primer
cgactgctctctgcctcaatttc

SEQ ID NO: 212 - DNA, 23 bp, Artificial Sequence
  Primer
cagcatctccatttcacaggaga

SEQ ID NO: 213 - DNA, 17 bp, Artificial Sequence
  Primer
cagcctccaggacaggc

SEQ ID NO: 214 - DNA, 20 bp, Artificial Sequence
  Primer
gggagccatgagcaagactc

SEQ ID NO: 215 - DNA, 19 bp, Artificial Sequence
  Primer
gtgctggagggtgacacag

SEQ ID NO: 216 - DNA, 19 bp, Artificial Sequence
  Primer
cccgaggacatccagacag

SEQ ID NO: 217 - DNA, 27 bp, Artificial Sequence
  Primer
gtggactcggtataaatcagatttctg

SEQ ID NO: 218 - DNA, 21 bp, Artificial Sequence
  Primer
ctggagcaggattaggtggtt

SEQ ID NO: 219 - DNA, 20 bp, Artificial Sequence
  Primer
gttccatctcctgctggtga

SEQ ID NO: 220 - DNA, 21 bp, Artificial Sequence
  Primer
gtgtgttcatgtgggttggac

SEQ ID NO: 221 - DNA, 20 bp, Artificial Sequence
  Primer
ctggtgtcctcgtcttgcca

SEQ ID NO: 222 - DNA, 25 bp, Artificial Sequence
  Primer
gtccaggagccatttcatatagaca

SEQ ID NO: 223 - DNA, 25 bp, Artificial Sequence
  Primer
ccacaaccaaatgagtctgcaaata

SEQ ID NO: 224 - DNA, 14 bp, Artificial Sequence
  Primer
tggtgtgtgtcctg

SEQ ID NO: 225 - DNA, 25 bp, Artificial Sequence
  Primer
ctgtcataggacagggtttaagact

SEQ ID NO: 226 - DNA, 26 bp, Artificial Sequence
  Primer
ccaccagactttaaaccagaggttta

SEQ ID NO: 227 - DNA, 28 bp, Artificial Sequence
  Primer
ggaagatgcaaagaaagaaagatactca

SEQ ID NO: 228 - DNA, 25 bp, Artificial Sequence
  Primer
gctttgaccctagtttttgttcgtg

SEQ ID NO: 229 - DNA, 28 bp, Artificial Sequence
  Primer
ctcaatcaataagaatatggccttggag

SEQ ID NO: 230 - DNA, 18 bp, Artificial Sequence
  Primer
cccagtggtgcttcagct

SEQ ID NO: 231 - DNA, 12 bp, Artificial Sequence
  Syntheticaly generated oligonucleotide
ggccggcgttcc

SEQ ID NO: 232 - DNA, 12 bp, Artificial Sequence
  Syntheticaly generated oligonucleotide
gtggggaaccgg

SEQ ID NO: 233 - DNA, 12 bp, Artificial Sequence
  Syntheticaly generated oligonucleotide
ttccggaatcga

SEQ ID NO: 234 - DNA, 12 bp, Artificial Sequence
  Syntheticaly generated oligonucleotide
ttccggaatcgg

SEQ ID NO: 235 - DNA, 12 bp, Artificial Sequence
  Syntheticaly generated oligonucleotide
ttccggaattgg

SEQ ID NO: 236 - DNA, 12 bp, Artificial Sequence
  Syntheticaly generated oligonucleotide
ttcgggaaccgg

SEQ ID NO: 237 - DNA, 12 bp, Artificial Sequence
  Syntheticaly generated oligonucleotide
ttcgggaatcgg

SEQ ID NO: 238 - DNA, 12 bp, Artificial Sequence
  Syntheticaly generated oligonucleotide
ttggggaaccgg

(19) United States
(12) Patent Application Publication
(10) Pub. No.: US 2010/0028868 A1
(43) Pub. Date: Feb. 4, 2010

(54) Responsiveness to Therapy for Liver Disorders

(76) Inventors: Yuchi Hwang, Taipei (TW); Kuang-Den Chen, Taipei (TW); Chingwei Chang, Taipei (TW); Jui-Lin Chen, Taipei (TW); Ding-Shinn Chen, Taipei (TW); Pei-Jer Chen, Taipei (TW); Ming-Yang Lai, Taipei (TW)

Correspondence Address: FISH & RICHARDSON PC, P.O. BOX 1022, MINNEAPOLIS, MN, 55440-1022, US

(21) Appl. No.: 12/181,928

(22) Filed: Jul. 29, 2008

(51) Int. Cl. C12Q 1/68

(52) U.S. Cl. 435/6

(57) ABSTRACT

Sets of nucleic acids and methods for predicting a subject's responsiveness to therapy for liver disorders.

### BACKGROUND

Liver disorders, such as hepatitis, are major public health concerns. For example, hepatitis C is estimated to affect 200 million people worldwide. Patients with liver damage resulting from hepatitis C may develop chronic liver diseases, such as cirrhosis and hepatocellular carcinoma. Hepatitis C can be treated with interferon α and ribavirin. However, interferon α or ribavirin therapy causes significant side effects and is expensive. More importantly, only about 50% hepatitis C patients are responsive to the treatment. New therapies have been vigorously sought. Although several drug candidates are now being evaluated, the progress is rather slow due to a lack of appropriate systems for determining a patient's response to new therapies. Thus, there is a need for a reliable system and method for predicting a patient's response to treatment of hepatitis C and other liver disorders.

A patient's response to viral therapy is associated with various viral factors, e.g., the viral level, viral genotype, and mutation in certain viral proteins, and host factors, e.g., the patient's age, gender, race, host immune response, HLA alleles, and other genetic compositions, such as polymorphisms.

Single nucleotide polymorphisms (SNPs), a set of single nucleotide variants at genomic loci, are distributed throughout a genome. An SNP can be “allelic.” More specifically, due to polymorphism, some members of a species have the unmutated sequence (i.e., the wild-type allele) and others have a mutated sequence (i.e., the mutant allele). In humans, a polymorphism or a set of polymorphisms may be associated with a genetic disorder. In addition, patients having different SNP genotypes respond to the same treatment differently. Therefore, an SNP genotype of a patient is expected to provide individualized guidance for preventing and treating various human disorders.

### SUMMARY

This invention relates to sets of target nucleic acids, primers, and methods that can be used to predict the responsiveness of a subject to a therapy for treating hepatitis C and other liver disorders. The target nucleic acids contain one or more SNPs. The primers can be used in polymerase chain reaction (PCR) amplification for obtaining sequences containing the SNPs or can be used in single-base-extension for SNP typing.

Accordingly, one aspect of this invention features a set of SNP-containing target nucleic acids that include one or more of (i) a first target nucleic acid obtained from amplification of the human Adenosine Deaminase-RNA-specific (ADAR) gene nucleic acid template with a first pair of primers, each containing an oligo-nucleotide selected from the ADAR gene region; (ii) a second target nucleic acid obtained from amplification of the human Caspase 5 (CASP5) gene nucleic acid template with a second pair of primers, each containing an oligo-nucleotide selected from the CASP5 gene region; (iii) a third target nucleic acid obtained from amplification of the human Fibroblast Growth Factor 1 (FGF1) gene nucleic acid template with a third pair of primers, each containing an oligo-nucleotide selected from the FGF1 gene region; (iv) a fourth target nucleic acid obtained from amplification of the human Interferon Consensus Sequence Binding Protein 1 (ICSBP1) gene nucleic acid template with a fourth pair of primers, each containing an oligo-nucleotide selected from the ICSBP1 gene region; (v) a fifth target nucleic acid obtained from amplification of the human Interferon-Induced Protein 44 (IFI44) gene nucleic acid template with a fifth pair of primers, each containing an oligo-nucleotide selected from the IFI44 gene region; (vi) a sixth target nucleic acid obtained from amplification of the human Phosphoinositide-3-Kinase Catalytic Gamma Polypeptide (PIK3CG) gene nucleic acid template with a sixth pair of primers, each containing an oligo-nucleotide selected from the PIK3CG gene region; (vii) a seventh target nucleic acid obtained from amplification of the human Transporter 2 ATP-Binding Cassette Sub-Family B (TAP2) gene nucleic acid template with a seventh pair of primers, each containing an oligo-nucleotide selected from the TAP2 gene region; and (viii) an eighth target nucleic acid obtained from amplification of the human Transforming Growth Factor, Beta Receptor Associated Protein 1 (TGFBRAP1) gene nucleic acid template with a eighth pair of primers, each containing an oligo-nucleotide selected from the TGFBRAP1 gene region.

A “target nucleic acid” refers to an isolated nucleic acid that contains one or more SNPs of interest. An isolated nucleic acid refers to a nucleic acid the structure of which is not identical to that of any naturally occurring nucleic acid or to that of any fragment of a naturally occurring genomic nucleic acid. The term therefore covers, for example, (a) a nucleic acid incorporated into a vector or into the genomic DNA of a prokaryote or eukaryote in a manner such that the resulting molecule is not identical to any naturally occurring vector or genomic DNA and (b) a separate molecule such as a cDNA, a genomic fragment, a fragment produced by polymerase chain reaction (PCR), or a restriction fragment. Specifically excluded from this definition are nucleic acids present in mixtures of different (i) DNA molecules, (ii) transfected cells, or (iii) cell clones: e.g., as these occur in a DNA library such as a cDNA or genomic DNA library.

Each of the above-mentioned target nucleic acids has a nucleotide at a single nucleotide polymorphism site and is 20-1,000 (e.g., 200-1,000) nucleotides in length. Each of the above-mentioned oligo-nucleotide is 10-50, such as 14-50 (e.g., 14-40), nucleotides in length. A primer containing such oligo-nucleotide is 10-60 (e.g., 12-55) nucleotides in length. Exemplary sequences of the target nucleic acids (SEQ ID NOs: 1-74) are listed in Table I below. Exemplary primers (SEQ ID NOs: 75-230), including forward (“F”) and reverse (“R”) primers, are listed in Table 2 below.

TABLE 1
Exemplary Target Nucleic Acids and Corresponding Primers

| | | | | | | Amplification | Amplification/ |
|---|---|---|---|---|---|---|---|
| | | | SEQ | | | Amplification | SNP |
| | SNP | SNP | ID | | | Primer | Primer |
| Gene | ID/public | ID/VGV | NO.: | Allele | SNP Flanking Sequence | ID | ID |
| ADAR | | VGV1473 | 1 | Y | TGGCGTGAACCCGGGAGGCGGAGCTTGCAGTGAG | O1081-F | |
| | | | | | CCGAGATCGCGCCACTGCACTCCAGC[C/T]TGA | | |
| | | | | C/T | GAGACAGATGATACTCCATCTCTAAATCAATCAA | O1082-R | |
| | | | | | TCAATCAATCAATCAATCAAT | | |
| ADAR | rs903323 | VGV1600 | 2 | Y | AGCTTCAGTGGTTTTGCTCTCTGTTATGCCCTTC | O1077-F | |
| | | | | | AGAAGCTCCTGTTTTCCTCAATCTGT[C/T]CAC | | |
| | | | | | TGGCCTATTAGGACCTACAGTGCAGGGCCTGACC | | |
| | | | | C/T | AGCTATCTGAGTGAGGTAAAGG | O1078-R | |
| ADAR | | VGV1798 | 3 | Y | CTGAGGCAGGAGAATGGCGTGAACCCGGGAGGCG | O1081-F | |
| | | | | | GAGCTTGCAGTGAGCCGAGATCGCGC[T/C]ACT | | |
| | | | | | GCACTCCAGCTTGAGAGACAGAGTGATACTC | | |
| | | | | C/T | CATCTCTAAATCAATCAATCAATCA | O1082-R | |
| ADAR | | VGV1449 | 4 | Y | CTCTGGCTGGGAGCAGTGGCTCACGCCTGTAATC | O1081-R | |
| | | | | | CCAGCACTTTGGGAGGCTGAGGTGGA[T/C]CTC | | |
| | | | | | ACGAGGTCAGGAGATTGAGACCATCCTGGCTA | | |
| | | | | C/T | ACACGGTGAAACCCCATCTCTACT | O1082-R | |
| ADAR | | VGV1464 | 5 | Y | GGGAAGGGCCCTGCTGGGTACGTAATCAAAAGGT | O1081-F | |
| | | | | | GCCTGATGAACCCCACCCCACCCAGA[C/T]GCA | | |
| | | | | | AATTTACCCACAAAGGGAGGTTCTTTGAAAT | | |
| | | | | C/T | GGCTCCTTTCCAAAGGCTGAGGGAC | O1082-R | |
| ADAR | rs1127309 | VGV1594 | 6 | Y | GATTATTTCTGCATGGCAGTCATTGACAGTTTCT | O1079-F | |
| | | | | | CCTTTTAGGCTGAGAGAATCTCCTTT[C/T]ACA | | |
| | | | | | CAGCGATTCCCTAGGAAGGTGTTTAAAACAGAA | | |
| | | | | C/T | ATAGAATAATGGAAGGAAACCGA | O1080-R | |
| CASP5 | rs518604 | | 7 | R | ATAACATTGCTCTTTGTAGGATCAAGTGGGATAT | O3101-F | |
| | | | | | ATGTAGAAGAGGGCTTGAAGTTGATC[A/G]TTT | | |
| | | | | | GGAAAGACAGCCAGTACTGGGATCCATAAAAC | | |
| | | | | A/G | TTCTATTCAAAATGTTAAATGGAT | O3102-R | O3185 |
| CASP5 | rs2282658 | | 8 | S | CAAGGTGGTCTCTAACAGGATGATGACATGTTTA | O3107-F | |
| | | | | | CTGAAAATGAGAGTTTAGAAATGAAA[C/G]TGT | | |
| | | | | | AGGTAGATCACAGATAACACTGCATGGGCCTT | | |
| | | | | C/G | GGAGTTGAATATATTCTGGAAAAT | O3108-R | O3188 |
| CASP5 | rs484345 | | 9 | R | TCCAAATAATACTTACAGTCAAGTGGCTGACTCC | O3157-F | |
| | | | | | TCCTATTTCATGGTCAACCAAATTGC[A/G]TCA | | |
| | | | | | TTATCATCATCAACATCACCACTATCATTGTTGT | | |
| | | | | A/G | CATCATTATCTTTATTGAGCAA | O3158-R | O3213 |
| CASP5 | rs1699087 | | 10 | M | TGCAGGTATCTGCAGCTACCTCCTTCCTGCCACA | O3159-F | O3214 |
| | | | | | ACCTCTGCTGATCAGAAAGGTTATTT[A/C]ATT | | |
| | | | | | TTGGAATTTAGTGCTCATTATATATGAGAATTG | | |
| | | | | A/C | TACGTGATAAATAATATATAATT | O3160-R | |
| CASP5 | rs2282657 | | 11 | R | CATGGGCCTTGGAGTTGAATATATTCTGGAAAAT | O3109-F | |
| | | | | | TTAACATATTTATCGTGTTAGATGCA[A/G]CCT | | |
| | | | | | TACGTTTTACACTGGTGATCTTTTGGTCCATATT | | |
| | | | | A/G | GAGAAGTGTTTGGGTAAACATT | O3110-R | O3189 |
| CASP5 | rs3181318 | | 12 | R | TGGTCATCCATTGTATTCAGATTTCTCTCTCTTG | O3113-F | O3805 |
| | | | | | CTCAAACTCATGATGACCTACCTGAA[A/G]TGT | | |
| | | | | | GTGCACCCAGGACAGTCCATTCTCTTGTCTAGAC | | |
| | | | | A/G | TGTAAATTATTCCTACTAGACT | O3114-R | O3805 |
| CASP5 | rs1790203 | | 13 | R | GGGGTCTAAATGAAAAACTTTGGGAGAAGAGCAA | O3129-F | O3199 |
| | | | | | CGTGCTCTGCACTGACCAGAAGAAAG[A/G]CAT | | |
| | | | | | TACTTCAGTATTTTCTGTATATGGCTTGATTA | | |
| | | | | A/G | TCCCTTATCCAAAATGCATGCTAC | O3131-R | O3199 |
| FGF1 | rs249926 | VGV567 | 14 | Y | GTTTCGCCCTTGTGAGGCACACTGGGCAATGCTG | O843-F | |
| | | | | | CCATTCCCATTCCACAGGTGAGGAAA[C/T]TGA | | |
| | | | | | GTCTCAGCGAGACTAAATGATTTTCCTGAAAA | | |
| | | | | C/T | TTATCTGGGAACACTAGAGACACT | O844-R | |
| FGF1 | | VGV679 | 15 | M | GAACCCTAGTAAATAGAAGTTTCGCCCTTGTGAG | O843-F | |
| | | | | | GCACACTGGGCAATGCTGCCATTCCC[A/C]TTT | | |
| | | | | | CCACAGGTGAGGAAACTGAGTCTCAGCGAGACT | | |
| | | | | A/C | AAATGATTTTCCTGAAAATTATCT | O844-R | |
| FGF1 | rs2282799 | VGV714 | 16 | M | GGGGGGCTTGAAGCTTCTTTCGCAGAGTTTGCAA | O843-F | |
| | | | | | ACAGAAAGAATGCATAATGGCAAGAA[A/C]GTT | | |
| | | | | | AATTGTCCAGGGCTGCTCCAGGTAGAAAGGGG | | |
| | | | | A/C | CAGAGTAGGCTTGAACTCGAGCCT | O844-R | |
| FGF1 | rs2282798 | | 17 | R | CAGGTGAGGAAACTGAGTCTCAGCGAGACTAAAT | O843-F | |
| | | | | | GATTTTCCTGAAAATTATCTGGGAAC[A/G]CTA | | |
| | | | | | GAGACACTTCAATTTCTAGTCAGGAAAGGACT | | |
| | | | | A/G | GGAAGCGTCCCAGGGCTGGGGGGC | O844-R | |
| FGF1 | | VGV676 | 18 | Y | GAGGCAATTCTGTTTCTGAATAACCCTTGAAACT | O843-F | |
| | | | | | CAGAAGGGCTCTGGCAGTACCACCAC[C/T]GGG | | |
| | | | | | CAGAAGAGGGCAACAGAACCACATTCAGGGA | | |
| | | | | C/T | GTACATCCGTGCCCAGGACTCCTCT | O844-R | |
| FGF1 | | VGV573 | 19 | M | TCACATTTAGTCAGGGAGCATCCGTCTCATGCCT | O843-F | |
| | | | | | GGCCGAGGCAATTCTGTTTCTGAATA[A/C]CCC | | |
| | | | | | TTGAAACTCAGAAGGGCTCTGGCAGTACCACCA | | |
| | | | | A/C | CTGGGCAGAAGAGGGCAACAGAA | O844-R | |
| FGF1 | | VGV700 | 20 | Y | CAAGGAATCAAAAATATCTCCTTGCCAGGCCGTG | O843-F | |
| | | | | | TGGATCACATTTAGTCAGGGAGCATC[C/T]GTC | | |
| | | | | | TCATGCCTGGCCGAGGCAATTCTGTTTCTGAAT | | |
| | | | | C/T | AACCCTTGAAACTCAGAAGGGCT | O844-R | |
| FGF1 | rs2282797 | VGV674 | 21 | Y | TTTCGATTGGCTTTTAGAAACGCTCTTTCTGAAG | O843-F | |
| | | | | | GAAGTCTTAACGTGTGACTCTGTCAC[C/T]TCA | | |
| | | | | | GTCTCTAATTATGCTCAAACTAGTGATCAAGGAA | | |
| | | | | C/T | TCAAAAATATCTCCTTGCCAGG | O844-R | |
| FGF1 | | VGV625 | 22 | S | TTTTCGATTGGCTTTTAGAAACGCTCTTTCTGAA | O843-F | |
| | | | | | GGAAGTCTTAACGTGTGACTCTGTCA[C/G]TTC | | |
| | | | | | AGTCTCTAATTATGCTCAAACTAGTGATCAAGGA | | |
| | | | | C/G | ATCAAAAATATCTCCTTGCCAG | O844-R | |
| ICSBP1 | rs385989 | | 23 | K | TGACGACATGTGCCCAAGGTGGTCGGGGCACAGC | O2740-F | O2789 |
| | | | | | CTGGTTTTATACATTTTAGGGAGACA[G/T]GAG | | |
| | | | | | ACATCAATCAATACATGTAAGAAGTACACTGG | | |
| | | | | G/T | TTCCATCCAGAAAGACGGGGACAG | O2741-R | |
| ICSBP1 | rs305067 | | 24 | S | AGTGATTGGCTCAGGAAGGGGCATGGGACTGAAT | O2744-F | |
| | | | | | AATGGCCAATGAGCTGAGAGGAAAAA[C/G]ACC | | |
| | | | | | TGCTGGGGCTTCCAGGAAGGTCCCTCCTTCC | | |
| | | | | C/G | TTGCCACCTGGTGTGTCCAGAGGAT | O2745-R | O2791 |
| ICSBP1 | rs305097 | VGV1824 | 25 | R | CACCAAGAGAACATGTTCTAGGAGGCAGGAAGAA | O2750-F | |
| | | | | | GCAGCTCGCAGATTCTTAAGTCTCAC[A/G]CCT | | |
| | | | | | AGAAACGGACACAGCATCACTTCTACCTATGC | | |
| | | | | A/G | TGCTGGTCAAAGCAGGCACAGAGC | O2751-R | O2794 |
| ICSBP1 | | VGV1826 | 26 | M | GATTCTTAAGTCTCACGCCTAGAAACGGACACAG | O2750-F | |
| | | | | | CATCACTTCTACCTATGCTGCTGGTC[A/C]AAG | | |
| | | | | | CAGGCACAGAGCCCACTGGATTGAAGAAAGAG | | |
| | | | | A/C | GCATAGACCCCCACCTTTAAAGGG | O2751-R | |
| ICSBP1 | rs305088 | | 27 | Y | CGTGCGAGGTGCTGCGCTCACAGTATTACTCATG | O2754-F | |
| | | | | | CACCTTTCTGATAAGAAAAGTGAAAA[C/T]GTG | | |
| | | | | | AAGTTGAAAACTGAAGACGCCCAGCAACTTC | | |
| | | | | C/T | CTGAATCCAGCCCTCCACGTCCTGC | O2755-R | O2796 |
| ICSBP1 | rs870614 | VGV1827 | 28 | R | ACTGGGAACCTCCATAGTTACCACATGCTGCGCT | O2756-F | |
| | | | | | GACTTCTCTAACACGCTTTGGCCAAT[A/G]ATG | | |
| | | | | | TTTCCCTAATCACAGCAGCTCCTCATTTAGAAT | | |
| | | | | A/G | GTGTTCTTATTTAGGATGCGTTC | O2757-R | O2797 |
| ICSBP1 | rs305061 | | 29 | Y | ATGAAAGTGCTGTTCTCATCACTTCCTATCCATG | O2748-F | O2793 |
| | | | | | GTCCATGCTGTCCGTGTGACTTACCA[C/T]GGT | | |
| | | | | | GGACGTTGACCTTGGCCACCTGGCTGGCTGTTG | | |
| | | | | C/T | GGTTCTCCACTGGAAGTTTACTC | O2749-R | |
| ICSBP1 | rs305095 | | 30 | Y | ACCTATGCTGCTGGTCAAAGCAGGCACAGAGCCC | O2752-F | O2795 |
| | | | | | ACTGGATTGAAGAAAGAGGCATAGAC[C/T]CCC | | |
| | | | | | ACCTTTAAAGGGCTGAGTGTCAGAGAACTTG | | |
| | | | | C/T | TGGCCAACATTAATCCACCACAACT | O2753-R | |
| IF144 | rs2148686 | VGV199 | 31 | K | AAGCCCTCAAATCCCATTCCTAATCTGATGAGTC | O857-F | |
| | | | | | TATGGACCAATTTGTGGAGGACAGTA[G/T]ATT | | |
| | | | | | AAATAGATCTGATTTTTGCCATCAATGTAAGGA | | |
| | | | | G/T | GGATAAAAACTTGCATACCAATT | O858-R | |
| IF144 | rs2070123 | VGV2188 | 32 | Y | GGAAAATATATATGATTTGCCACTAGATCAAGAA | O4631-F | O4650 |
| | | | | | GTATGGCAGTGACAACTCGTTTGACA[C/T]GGT | | |
| | | | | | TGCACGAAAAGATCCTGCAAAATCATTTTGGAG | | |
| | | | | C/T | GGAAGCGGCTTAGCCTTCTCTAT | O4632-R | O7081 |
| IF144 | rs273249 | VGV2191 | 33 | R | CCTCAGTATAAAAGCTCTATCAGTACCATGAACA | O4637-F | |
| | | | | | ATCTCATCATAATCACACTTAATATC[A/G]TTT | | |
| | | | | | CATATAATCACAATCAAAACTGGAAACAATTAA | | |
| | | | | A/G | AACATTTTAGCATATTTTTATAG | O4638-R | O4653 |
| IF144 | | VGV33 | 34 | R | AGGTGGTTTCATTTGAGGCCTCATTTGTTACCAT | O861-F | |
| | | | | | TGAAATCAATGAAGGTGACTCCCCAT[G/A]TCA | | |
| | | | | | GAGAAATTCCAGATACTAATAAGTAGTCCAGG | | |
| | | | | A/G | GGAGTTTTTTGGGGAGATGAGGGT | O862-R | |
| IF144 | | VGV2189 | 35 | S | CTGTCTGCCTTGAGAACTTATGAACCATATGGAT | O4635-F | |
| | | | | | CCCTGGTTCAACAAATACGAATTCTG[C/T]TGC | | |
| | | | | | TGGGTCCAATTGGAGCTGGGAAGTCCAGCTTTT | | |
| | | | | C/G | TCAACTCAGTGAGGTCTGTTTTC | O4636-R | |
| IF144 | | VGV205 | 36 | Y | GGTCCAATTGGAGCTGGGAAGTCCAGCTTTTTCA | O859-F | |
| | | | | | ACTCAGTGAGGTCTGTTTTCCAAGGG[C/T]ATG | | |
| | | | | | TAACGCATCAGGCTTTGGTGGGCACTAATACAA | | |
| | | | | C/T | CTGGGATATCTGAGAAGGTAAGC | O860-R | |
| IF144 | | VGV2187 | 37 | M | GGGGTGTACAAATTATTGTATTTTAAAGTCAATC | O4637-F | |
| | | | | | AGAATAGTTTATTCTTGTATTATAAC[C/A]ATA | | |
| | | | | | ACAGTTCACTAATTAAATTAAATTTAGGAATTG | | |
| | | | | A/C | AATTGTTAAGTTAATTTGGTTTT | O4638-R | |
| IF144 | rs2296718 | VGV96 | 38 | K | CATAAATTTTAGTTACCTCTTCCAAGAGGTGGTT | O861-F | |
| | | | | | TCATTTGAGGCCTCATTTGTTACCAT[G/T]GAA | | |
| | | | | | ATCAATGAAGGTGACTCCCCATGTCAGAGAAAT | | |
| | | | | G/T | TCCAGATACTAATAAGTAGTCCA | O862-R | |
| IF144 | rs2296717 | VGV225 | 39 | Y | TCCAAGAGGTGGTTTCATTTGAGGCCTCATTTGT | O861-F | |
| | | | | | TACCATTGAAATCAATGAAGGTGACT[C/T]CCC | | |
| | | | | | ATGTCAGAGAAATTCCAGATACTAATAAGTAGT | | |
| | | | | C/T | CCAGGGGAGTTTTTTGGGGAGAT | O862-R | |
| IF144 | rs1051047 | | 40 | R | ACATTGTAGTACTTGTAAATAACTAGAAATAACA | O4647-F | |
| | | | | | TGATTTAGTCATAATTGTGAAAAATA[A/G]TAA | | |
| | | | | | TAATTTTTCTTGGATTTATGTTCTGTATCTGTG | | |
| | | | | A/G | AAAAAATAAATTTCTTATAAAAC | O4648-R | |
| PIK3CG | rs1526083 | | 41 | R | TGTTATCAATGGAAGCCTTCTCAAAAGGAATTGA | O2378-F | |
| | | | | | TTTGCATATGCACAGGCACTCCATTC[A/G]GTT | | |
| | | | | | GTCATCAAATGCCCTTTGTTCAGAGCTTCATCAT | | |
| | | | | A/G | CGGCAAAAGTAGATATGATGAA | O2379-R | O2426 |
| PIK3CG | rs3779501/IM | | 42 | Y | TGCTGCTTTTAAAATTATGAACTATTTCAAACTT | O2382-F | O2428 |
| | S-JST135578 | | | | ACAGAAAAGCACAGAGAACAATGAAA[C/T]ACC | | |
| | | | | | TATGCACTCACAAGATTTAATTGTGTTTTTTACA | | |
| | | | | C/T | TTTTATCAGTTCTTCCTCATCA | O2383-R | |
| PIK3CG | rs2037718 | | 43 | S | GATCCTATTTACAGCATTCTATTTATTAATTTTT | O2396-F | |
| | | | | | ATAAAAACCTAGTTTATTAAAAACTA[C/G]TTA | | |
| | | | | | CAGTAATATTTGATTTTTTAAAGACAATTAGGTC | | |
| | | | | C/G | ATTTGTAAATAATAAGTTTTCC | O2397-R | O2345 |
| PIK3CG | rs3173908 | | 44 | Y | GCTAGGATTATTTGCAGGTTTGGTTTTTTCTCAT | O2400-F | O2437 |
| | | | | | TTGTCTGTGGCATTGGAGAATATTCT[C/T]GGT | | |
| | | | | | TTAAACAGACTAATGACTTCCTTATTGTCCCTGA | | |
| | | | | C/T | TATTTTGACTATCTTACTATTG | O2401-R | O3264 |
| TAP2 | rs1871665 | | 45 | Y | GGAAAAGAGTAATGATTCTGGAAAGAAAGGTGAT | O2882-F | O2821 |
| | | | | | AAGCCTCAGAGTAAGATCTTCAGGGA[C/T]TAG | | |
| | | | | | CAAGATGAGCTGGGAAAGAAGAGTGAGAGG | | |
| | | | | C/T | GAGAAGCATACCCATCCTGAGAGAGT | O2883-R | |
| TAP2 | rs2071543 | | 46 | K | CCTCCACTCCTCAGCGCCCGCCTCCCTGCATCCC | O2888-F | O2824 |
| | | | | | TAGGGGCTTCCCTACTGCCCCGACCT[G/T]CAT | | |
| | | | | | TCCCCGGGGTAAAGCGAGCTCTGGAGATCGCAT | | |
| | | | | G/T | AGAGAAACTGTAGTGTCCTGGGT | O2889-R | |
| TAP2 | rs1800453 | | 47 | Y | AATGCTCGGGCCAACGCCACTGCCTGTCGCTGAC | O2892-F | O2826 |
| | | | | | CCCCTGACAGCTGGCTCCCAGCCTCG[C/T]CTA | | |
| | | | | | CCTCTGCAGAGCAAAGGGCCAAGATGAGAACG | | |
| | | | | C/T | GTATAGCCACATGTGTGCACGCAT | O2893-R | |
| TAP2 | rs1894407 | | 48 | M | AACTGTGAGAAATAAATTTCTGTTCTTCATAAAT | O2866-F | O2813 |
| | | | | | TACCCTATCTCGTGTATCTTGTTACA[A/C]TAA | | |
| | | | | | CACAAATGGACTAAGACAGAGAGCATAAGGCT | | |
| | | | | A/C | TGGGGGAAGAAGGGTACACTTCTT | O2867-R | |
| TAP2 | rs2857101 | | 49 | Y | TCAGATCATCTTCTTCTGTGAGGGCTGCAGCTTC | O2872-F | O2816 |
| | | | | | CATGTAGTTGGGAGATACAGGAATTA[C/T]TAT | | |
| | | | | | TCCTGTTTTATGAATAAAGGACATTTGTGGGAG | | |
| | | | | C/T | AGAAAGGAATCAGGCCAGAGTTC | O2873-R | |
| TAP2 | rs183585 | | 50 | R | CCTGATTTAAGCAAAGTATAATAAACACACTCAT | O2876-F | O2818 |
| | | | | | ACACATATACTACATGGATACCACAA[A/G]TG | | |
| | | | | | GAAATTTGACAATTGACTATTTGATAAATTTTA | | |
| | | | | A/G | AGAACTACTGTTAATTTTTTGGTG | O2877-R | |
| TAP2 | rs991760 | | 51 | R | TCATAGTCAATTACTCTGTGTTGGGTCTACACCA | O2900-F | |
| | | | | | CATCTGCACATACTATGAGCCCTTCC[A/G]TTG | | |
| | | | | | GAGATAATTTTCACTTGCGGAGCTGCTTCACTT | | |
| | | | | A/G | CTACCTGTAGGAGCCTCATCTCC | O2901-R | O2830 |
| TAP2 | rs1383269 | | 52 | R | ATTGGCTCATACTACTGTGGGAGCTGGCACGGTC | O2910-F | O2835 |
| | | | | | GAAATCTGCAGGTAGGCTGGAGACCC[A/G]GG | | |
| | | | | | AAGAGCTGATGTTGCGGCTTGAGTCTGAAGGTG | | |
| | | | | A/G | GTCCAGAGGCAGAATTCCCTCTTC | O2911-R | |
| TGFBRAP1 | rs2241796 | VGV2200 | 53 | Y | GTGAAGTGACAACAGCTTCAGCTCGTCATCGATG | O4719-F | O4781 |
| | | | | | TTATACAGAAACACAAAAGGAATCCC[C/T]GGC | | |
| | | | | | CTGTGATGAAGGAGAGGCCGTTGCTGTGTGTT | | |
| | | | | C/T | CAGGACACCTCAGAGCAGGCACAT | O4720-R | O7086 |
| TGFBRAP1 | | VGV2192 | 54 | R | CAGAAACACAAAAGGAATCCCCGGCCTGTGATGA | O4719-F | |
| | | | | | AGGAGAGGCCGTTGCTGTGTGTTCAG[G/A]ACA | | |
| | | | | | CCTCAGAGCAGGCACATAAAGTGCTGGAGG | | |
| | | | | A/G | GTGACACAGCCTGTCTGGATGTCCTC | O4720-R | |
| TGFBRAP1 | rs1866040 | VGV2204 | 55 | R | GGAGGGTGTGCCATCCAGGAGGCGACACCCCCAT | O4719-F | |
| | | | | | CCAGCACACGGGCCCTTCCACCCGCT[A/G]TCG | | |
| | | | | | GTCCTGCTAAAGGTACGAGGCTAAAACCGGC | | |
| | | | | A/G | CTCTCCAGAAAAGAACGCTCAGTGT | O4720-R | |
| TGFBRAP1 | | VGV2197 | 56 | Y | AAAACTGTCATGATGACAAAATGCAAGCATGACG | O4723-F | O7088 |
| | | | | | TAAAATGCCTAGGTCAGTGCCTGGCA[C/T]ACA | | |
| | | | | | GCACATGCTGGGTAAGCGCCTGATATTCTGA | | |
| | | | | C/T | TGCTGCTCTCCTCCCTACAGACTCT | O4724-R | O4783 |
| TGFBRAP1 | rs2576737 | VGV2325 | 57 | Y | CTTCCCTCTAGCCAACAGGTGCTTTTTCACTGCC | O4729-F | O7089 |
| | | | | | AGCATTTCTCAGCCTCCAGGACAGGC[C/T]GAG | | |
| | | | | | TCTTGCTCATGGCTCCCCTCCCTCCTCCAGGCCC | | |
| | | | | | A | | |
| | | | | C/T | CAAGCTCCATGTTGGCAGTGG | O4730-R | O7089 |
| TGFBRAP1 | rs1866039 | | 58 | Y | CGTTGCTGTGTGTTCAGGACACCTCAGAGCAGGC | O4721-F | O4782 |
| | | | | | ACATAAAGTGCTGGAGGGTGACACAG[C/T]CTG | | |
| | | | | | TCTGGATGTCCTCGGGAGGGTGTGCCATCCAG | | |
| | | | | C/T | GAGGCGACACCCCCATCCAGCACA | O4722-R | O7087 |
| TGFBRAP1 | | VGV2196 | 59 | M | AATGTGGGGTTCTCAACTGGCCTGGGAGGCAGCT | O4723-F | |
| | | | | | CTGGGCACGCCCATTTCCTGAGCATG[A/C]GAC | | |
| | | | | | TGCTCTCTGCCTCAATTTCCTCTCCTGTGAAATG | | |
| | | | | A/C | GAGATGCTGACAGTAAATACTG | O4724-R | |
| TGFBRAP1 | | VGV2203 | 60 | Y | CGTAAAATGCCTAGGTCAGTGCCTGGCACACAGC | O4723-F | |
| | | | | | ACATGCTGGGTAAGCGCCTGATATTC[T/C]GAT | | |
| | | | | | GCTGCTCTCCTCCCTACAGACTCTCTTAATCACC | | |
| | | | | C/T | AGCGTCCAAAAGGGGAGAAAAA | O4724-R | |
| TGFBRAP1 | hCV2099139 | | 61 | R | GACAATACTGAGATCATCCTCACCGAGAAGAAAG | O4727-F | O4785 |
| | | | | | TGGACTCGGTATAAATCAGATTTCTG[A/G]AGC | | |
| | | | | | AGCCGCCGCAGCTTGGCCTGCGTCTCGGTGG | | |
| | | | | A/G | CCTCTGCACCCTTGCCACTGGCGGA | O4728-R | |
| TGFBRAP1 | rs2576738 | | 62 | M | AGGGCCCAGTGTGGCCCAGGTCTGGGTGCTTCCT | O4731-F | |
| | | | | | CCTCCCAAAGATCAAGTCCTTCAGGG[A/C]AAC | | |
| | | | | | CACCTAATCCTGCTCCAGAAAACAGCAGTGTC | | |
| | | | | A/C | AGACTTCTGAGGGGTCGAGGAGCG | O4732-R | O4787 |
| TGFBRAP1 | rs2241800 | VGV326 | 63 | Y | TGAAGAGAATACCCAGCTGAGCCTGGAAACCAAG | O619-F | |
| | | | | | GCAGAAAAGCAACACCCAGGATAACA[C/T]GCC | | |
| | | | | | ATCAGAGTCTGCGCAAAGGCACCATCACAGC | | |
| | | | | C/T | TCTGCTGAAACCAGCATTTTCCTGG | O620-R | |
| TGFBRAP1 | rs1020063 | VGV308 | 64 | Y | AAAGCAACACCCAGGATAACACGCCATCAGAGTC | O619-F | |
| | | | | | TGCGCAAAGGCACCATCACAGCTCTG[C/T]TGA | | |
| | | | | | AACCAGCATTTTCCTGGCACTAAATTACAAAC | | |
| | | | | C/T | AGATTTGTTGAATGGTCCTTGAAG | O620-R | |
| TGFBRAP1 | rs1020064 | VGV296 | 65 | Y | AGAAATATTCATGTGGCCATTTCTGTGGGGACTC | O619-F | |
| | | | | | TCGTAGAATTTCAGAGCCTAACATTG[G/T]AAT | | |
| | | | | | GCAACAAACAGTTCCTTTCCCATCTCCTCTCCG | | |
| | | | | C/T | GAACCTCCTTGTCCTGGCTACAA | O620-R | |
| TGFBRAP1 | rs2576741 | VGV419 | 66 | Y | GAATGGCGAGGAAGGACCCAGGATCCATGCTGCC | O619-F | |
| | | | | | TGCGATGTTCAGAGCATCCTCCTTCA[C/T]CCA | | |
| | | | | | AGCTCTGATCAGCTGTCCTCCTCTACTGGCTTC | | |
| | | | | C/T | CACCTCTGGCTGCCCTTCCTTCC | O620-R | |
| TGFBRAP1 | rs2576743 | | 67 | Y | CACCCAGGCTTTCTTAGTCAGAGATGCAGAAATG | O4741-F | O4792 |
| | | | | | CTGCATGTTCCATCTCCTGCTGGTGA[C/T]GTC | | |
| | | | | | CAACCCACATGAACACACCAACATCCGACACTC | | |
| | | | | C/T | CTGCAATAAAGGGGCCAGTTTAT | O4742-R | O7090 |
| TGFBRAP1 | rs3792047/IM | | 68 | Y | TCTCTGATCCCACGCCCCACTCCGTCACCGTCCA | O4743-F | O4793 |
| | S-JST149411 | | | | GCATGGCTGGTGTCCTCGTCTTGCCA[C/T]TGT | | |
| | | | | | CTATATGAAATGGCTCCTGGACATGTTTCTTCA | | |
| | | | | C/T | CTCTGTTCCTACAGATGCCAACA | O4744-R | O7091 |
| TGFBRAP1 | rs920217 | | 69 | Y | CAGCTATAGTAAGAATTTTTTGAGAAAGATGACA | O4747-F | O4795 |
| | | | | | ACCACAACCAAATGAGTCTGCAAATA[C/T]CAC | | |
| | | | | | AGCAGGACACACACCATGTAAACCCTGGAGC | | |
| | | | | C/T | TGAGGGAAGATGAACAGGCACACGG | O4748-R | O7093 |
| TGFBRAP1 | rs2576750 | | 70 | W | AGAGTAGCTGAATTATTTACCTTCTTCAAGAAGC | O4757-F | O4800 |
| | | | | | ACTGTCATAGGACAGGGTTTAAGACT[A/T]TAA | | |
| | | | | | ACCTCTGGTTTAAAGTCTGGTGGTTACTATGC | | |
| | | | | A/T | TGAAGATAGAATCTGTATATAGGT | O4758-R | O7094 |
| TGFBRAP1 | | VGV2199 | 71 | W | GAATTTAACAACGTAATAATCCCTAAAGGAAAAC | O4771-F | |
| | | | | | ACACTGCCTCCTTCTCATCTGTCGCT[A/T]GTC | | |
| | | | | | CATATGAAAAGTAGCAACGTCTGGTGGACAA | | |
| | | | | A/T | GGGCAGCCTCAGAATGGGCTCTGGG | O4772-R | |
| TGFBRAP1 | rs1561237 | | 72 | R | CAGATTCCCACACACTGGACTTCCTCTGACTGGC | O4771-F | |
| | | | | | TGATGGCAGGGATCCTGGCTTTAGGC[A/G]TGA | | |
| | | | | | GTATCTTTCTTTCTTTGCATCTTCCTACAATTT | | |
| | | | | A/G | ACAGTTTTTGTATAAATACAATG | O4772-R | O4807 |
| TGFBRAP1 | hCV2099090 | | 73 | W | CTCAAGGTTACCTGGCTACAAGGTGATGAAACAG | O4775-F | O4809 |
| | | | | | GGCTTTGACCCTAGTTTTTGTTCGTG[A/T]CTC | | |
| | | | | | CAAGGCCATATTCTTATTGATTGAGGGGAACAA | | |
| | | | | A/T | GTATGATTTCAGATCCTCTCCTA | O4776-R | O7095 |
| TGFBRAP1 | rs2246094 | | 74 | S | CCCCTACCCTAGTGTGACTCATGGCCACATAAAT | O4779-F | O4811 |
| | | | | | GCCCCATCCCCAGTGGTGCTTCAGCT[C/G]TGA | | |
| | | | | | CCCAGTGGAACCAGTGTCACCGGCTCAGCCTCC | | |
| | | | | C/G | AGGTAGGGGACTGGCTTCCCAGG | O4780-R | |

Notes:
1. SNPID/public: SNP ID in public database dbSNP in the Entrez system of the National Center for Biotechnology Information at http://www.ncbi.nlm.nih.gov/SNP/index.html (started with “rs (reference SNP)”) and in the SNP database of Celera Genomics (started with “hCV”).
2. SNPID/VGV: SNP ID in the database of Vita Genomics, Taipei, Taiwan.
3. Amplification Primer: a primer that can be used in PCR amplification (same for Table 2 below).
4. Amplification/SNP Primer: a primer that can be used in PCR amplification and in single-base-extenstion-based SNP typing (same for Table 2 below).
5. Each SNP is bracketed.

TABLE 2
Amplification Primers and corresponding Amplification/SNP Primers

| | | | Amplification | | |
|---|---|---|---|---|---|
| Amplification | SEQ | | SNP/Primer | SEQ | |
| Primer ID | ID NO.: | Primer Sequences | ID | ID NO.: | Primer Sequences |
| O1081-F | 75 | GAGGTGAAAATGGGAACAAAGG | | | |
| O1082-R | 76 | AAAACTACAACCAAGCCTGTCC | | | |
| O1077-F | 77 | TAAGCCGTTTTCTGAGAGAGGTG | | | |
| O1078-R | 78 | CTCTTTGCTCAGTCTGGGATTT | | | |
| O1079-F | 79 | GGAGAGAATATGGGAGTCTAGGA | | | |
| O108O-R | 80 | GCATAGTTAGCCTTTTGGGTCTC | | | |
| O3101-F | 81 | TTCACATTTTTATCTGGACACTT | | | |
| O3102-R | 82 | ACACTTTACAGTCAAAGGCATAC | O3185 | 177 | CCAGTACTGGCTGTCTTTCCAAA |
| O3107-F | 83 | CATGTTTACTGAAAATGAGAGTTT | | | |
| O3108-R | 84 | ATGATACCAAAATTGAAGACAAG | O3188 | 178 | GCAGTGTTATCTGTGATCTACCTACA |
| O3157-F | 85 | GACTCCTCCTATTTCATGGTC | | | |
| O3158-R | 86 | GCCCCATACTTGATATTAGATGT | O3213 | 179 | GTGGTGATGTTGATGATGATAATGA |
| O3159-F | 87 | GAGAATCCAAATGCAGATAGAG | O3214 | 180 | CCTCTGCTGATCAGAAAGGTTATTT |
| O3160-R | 88 | ATGTTTTTCAGGGATAGACAAA | | | |
| O3109-F | 89 | CATGTTTACTGAAAATGAGAGTTT | | | |
| O3110-R | 90 | ATGATACCAAAATTGAAGACAAG | O3189 | 181 | AAAAGATCACCAGTGTAAAACGTAAGG |
| O3113-F | 91 | TCCATTGTATTCAGATTTCTCTC | O3191 | 182 | CTCAAACTCATGATGACCTACCTGAA |
| O3114-R | 92 | TAAGCACTTTTAAGCATTTTGAG | O3805 | 183 | CTGTCCTGGGTGCACACA |
| O3129-F | 93 | AAGGGGTCTAAATGAAAAACTT | O3807 | 184 | GCTCTGCACTGACCAGAAGAAAG |
| O3130-R | 94 | CACATGATATTCAAAGGAAATGT | O3199 | 185 | TCAAGCCATATACAGAAAATACTGAAGTAATG |
| O843-F | 95 | AAGAACAAGGACCAAGAGGACAG | | | |
| O844-R | 96 | CTTGTAGAGCTCAAAGGCATACG | | | |
| O2740-F | 97 | AGGTTTATTCGACAAAGTTAAGG | O2789 | 186 | GCCTGGTTTTATACATTTTAGGGAGACA |
| O2741-F | 98 | GGTTGTCTCTAAACTCAAAAGAA | | | |
| O2744-F | 99 | ACTGAATAATGGCCAATGAG | | | |
| O2745-R | 100 | AGGTTCCTTGACTCCAACTC | O2791 | 187 | TGGAAGCCCCAGCAGGT |
| O2750-F | 101 | AGCAGCATAAATGGACTTCTT | | | |
| O2751-R | 102 | CAGTTGTGGTGGATTAATGTT | O2794 | 188 | TGATGCTGTGTCCGTTTCTAGG |
| O2754-F | 103 | TCCACACCTCAAGGTAGTTC | | | |
| O2755-R | 104 | GTGAGGTGAGATCCTGAAAC | O2796 | 189 | GGCGTCTTCAGTTTTCAACTTCAC |
| O2756-F | 105 | GACTGGGAACCTCCATAGTTA | | | |
| O2757-R | 106 | GTATCCTACGGGACAGAGTTTAT | O2797 | 190 | AGCTGCTGTGATTAGGGAAACAT |
| O2748-F | 107 | GCTTTTCTGATGTTTTTCTCAT | O2793 | 191 | GCTGTCCGTGTGACTTACCA |
| O2749-R | 108 | TCTTTAATGGATTTAGTGAGTCTG | | | |
| O2752-F | 109 | GGACACAGCATTACTTCTACCTA | O2795 | 192 | ACTGGATTGAAGAAAGAGGCATAGAC |
| O2753-R | 110 | ACTCCACAGAAAAGATCAATGT | | | |
| O857-F | 111 | ATGCTAAGGTACCCACAAGATGG | | | |
| O858-R | 112 | CTTCTTGGCCATGGGAATTTG | | | |
| O4631-F | 113 | TTTGCCACTAGATCAAGAAGTAT | O4650 | 193 | GGCAGTGACAACTCGTTTGACA |
| O4632-R | 114 | CATACTTTCCTTCCTGGTAACTC | O7081 | 194 | GCAGGATCTTTTCGTGCAACC |
| O4637-F | 115 | ATCAGTACCATGAACAATCTCAT | | | |
| O4638-R | 116 | AAACCAAATTAACTTAACAATTCAA | O4653 | 195 | AATTGTTTCCAGTTTTGATTGTGATTATATGAAA |
| O861-F | 117 | TAGAGCCCTTTGGTCTTTAGTC | | | |
| O862-R | 118 | CTGGGCAACAGAAACTTCATC | | | |
| O4635-F | 119 | CCTAACCTCATCAATTGTTAAA | | | |
| O4636-R | 120 | AAATGTGCTTACCTTCTCAGATA | | | |
| O859-F | 121 | AAACAGGCTCAGGAAGAGCTTA | | | |
| O860-R | 122 | CTGCCTTCAGACTTCCTCTTACA | | | |
| O4647-F | 123 | AGTATCTAAGACCAAAGGGATGT | | | |
| O4648-R | 124 | AAAGCAGTGAATATCAGAAGATG | | | |
| O2378-F | 125 | GCATTACCTTCTACAATTGGTC | | | |
| O2379-R | 126 | GTGAAGTACTAATGTCTGCAAAAC | O2426 | 196 | GAACAAAGGGCATTTGATGACAAC |
| O2382-F | 127 | AAAGGATACACCATTTCTATTCA | O2428 | 197 | ACAGAAAAGCACAGAGAACAATGAAA |
| O2383-R | 128 | TGAGGAAGAACTGATAAAATGTAA | | | |
| O2396-F | 129 | GCATAGGTTCTACATGTTTTATGT | | | |
| O2397-R | 130 | AATCAGAAAAGTGAAGGAAAACT | O2435 | 198 | ATGACCTAATTGTCTTTAAAAAATCAAATATTACTGT |
| | | | | | AA |
| O2400-F | 131 | CTCTTTTCCTACCTGAACTCTTC | O2437 | 199 | TGTCTGTGGCATTGGAGAATATTCT |
| O2401-R | 132 | TAAACCCAGATGAAAATAGATGT | O3264 | 200 | ACAATAAGGAAGTCATTAGTCTGTTTAAACC |
| O2882-F | 133 | AGTGAAGGGAAAAGAGTAATGAT | O2821 | 201 | GCCTCAGAGTAAGATCTTCAGGGA |
| O2883-R | 134 | TTAAGTCTCAGATGGAATGTCTC | | | |
| O2888-F | 135 | CTAACTTGCACTTCCTCCTCT | O2824 | 202 | CCCTACTGCCCCGACCT |
| O2889-R | 136 | AGGACACTACAGTTTCTCTATGC | | | |
| O2892-F | 137 | CATCATCCAGGATAAGTACACAC | O2826 | 203 | CTGGCTCCCAGCCTCG |
| O2893-R | 138 | TTCTGTTCATCTTCCCAGAAT | | | |
| O2866-F | 139 | GGTATCATGGTAACCACAAGTT | O2813 | 204 | AATTACCCTATCTCGTGTATCTTGTTACA |
| O2867-R | 140 | AATCGTCCTGTCTTTATTCTTTT | | | |
| O2872-F | 141 | TAGGTTTGACTGTTGCATTATTT | O2816 | 205 | CATGTAGTTGGGAGATACAGGAATTA |
| O2873-R | 142 | AATTTCACAGAGGGTAAAATAGG | | | |
| O2876-F | 143 | TATTCTGAAGCAAATCCAAGATA | O2818 | 206 | TCTTAAAATTTATCAAATAGTCAATTGTCAAATTTCC |
| | | | | | A |
| O2877-R | 144 | AGTCACAATATGTGGACCATTT | | | |
| O2900-F | 145 | CAATTACTCTGTGTTGGGTCTAC | | | |
| O2901-R | 146 | TCATCTTTCCACTTTCTCTATTG | O2830 | 207 | CTCCGCAAGTGAAAATTATCTCCAA |
| O2910-F | 147 | ATAGATTTTGGGTAAAGCAGATT | O2835 | 208 | GCCGCAACATCAGCTCTTCC |
| O2911-R | 148 | TTGGCAGAGAGAGATTTATTTTA | | | |
| O4719-F | 149 | TCGATGTTATACAGAAACACAAA | O4781 | 209 | TGTTATACAGAAACACAAAAGGAATCCC |
| O4720-R | 150 | ACACACTGAGCGTTCTTTTC | O7086 | 210 | CCTCTCCTTCATCACAGGCC |
| O4723-F | 151 | AGAATGTGGGGTTCTCAACT | O7088 | 211 | CGACTGCTCTCTGCCTCAATTTC |
| O4724-R | 152 | ATTAAGAGAGTCTGTAGGGAGGA | O4783 | 212 | CAGCATCTCCATTTCACAGGAGA |
| O4729-F | 153 | ATACTGTCCTGAGCCTGCTA | O4786 | 213 | CAGCCTCCAGGACAGGC |
| O4730-R | 154 | AAGTCTGACACTGCTGTTTTCT | O7089 | 214 | GGGAGCCATGAGCAAGACTC |
| O4721-F | 155 | TCGATGTTATACAGAAACACAAA | O4782 | 215 | GTGCTGGAGGGTGACACAG |
| O4722-R | 156 | ACACACTGAGCGTTCTTTTC | O7087 | 216 | CCCGAGGACATCCAGACAG |
| O4727-F | 157 | AAGGGAGACAATACTGAGATCAT | O4785 | 217 | GTGGACTCGGTATAAATCAGATTTCTG |
| O4728-R | 158 | CTTTGACTTTCCCACTTGAA | | | |
| O4731-F | 159 | ATACTGTCCTGAGCCTGCTA | | | |
| O4732-R | 160 | AAGTCTGACACTGCTGTTTTCT | O4787 | 218 | CTGGAGCAGGATTAGGTGGTT |
| O619-F | 161 | GGTGTAGGGTAGCTTGAGATCAG | | | |
| O620-R | 162 | AAAAGAACAGAAGGGACGAAGG | | | |
| O4741-F | 163 | AACTCACAGTACCACCTTTCAG | O4792 | 219 | GTTCCATCTCCTGCTGGTGA |
| O4742-R | 164 | GATGTAACAAGAACAAAGTGGTC | O7090 | 220 | GTGTGTTCATGTGGGTTGGAC |
| O4743-F | 165 | GTTCTTGTTACATCCAGCAATAG | O4793 | 221 | CTGGTGTCCTCGTCTTGCCA |
| O4744-R | 166 | GGTGTCAAAGTCAGAGAGAGTAA | O7091 | 222 | GTCCAGGAGCCATTTCATATAGACA |
| O4747-F | 167 | TCAGACTAGAATATTTTTCACAGC | O4795 | 223 | CCACAACCAAATGAGTCTGCAAATA |
| O4748-R | 168 | TGTCCCTGAATTAGCCATAG | O7093 | 224 | TGGTGTGTGTCCTG |
| O4757-F | 169 | GCTGAATTATTTACCTTCTTCAA | O4800 | 225 | CTGTCATAGGACAGGGTTTAAGACT |
| O4758-R | 170 | AATCTTTATGATGCACTGTGTCT | O7094 | 226 | CCACCAGACTTTAAACCAGAGGTTTA |
| O4771-F | 171 | GGATCGAATTTAACAACGTAATA | | | |
| O4772-R | 172 | CAAAAACTGTAAATTGTAGGAAGA | O4807 | 227 | GGAAGATGCAAAGAAAGAAAGATACTCA |
| O4775-F | 173 | TGGAGATACAGAGATCTTAGGTG | O4809 | 228 | GCTTTGACCCTAGTTTTTGTTCGTG |
| O4776-R | 174 | GAAGCAATAGCAAAGACTCCT | O7095 | 229 | CTCAATCAATAAGAATATGGCCTTGGAG |
| O4779-F | 175 | CAACAATCTTCCTGTGACTTG | O4811 | 230 | CCCAGTGGTGCTTCAGCT |
| O4780-R | 176 | GCTCTTGTACCTTCCCAATC | | 231 | |

In one embodiment of the set of target nucleic acids of this invention, (i) the first target nucleic acid contains SEQ ID NO: 1, 2, 3, 4, 5, or 6; (ii) the second target nucleic acid contains SEQ ID NO: 7, 8, 9, 10, 11, 12, or 13; (iii) the third target nucleic acid contains SEQ ID NO: 14, 15, 16, 17, 18, 19, 20, 21, or 22; (iv) the fourth target nucleic acid contains SEQ ID NO: 23, 24, 25, 26, 27, 28, 29, or 30; (v) the fifth target nucleic acid contains SEQ ID NO: 31, 32, 33, 34, 35, 36, 37, 38, 39, or 40; (vi) the sixth target nucleic acid contains SEQ ID NO: 41, 42, 43, or 44; (vii) the seventh target nucleic acid contains SEQ ID NO: 45, 46, 47, 48, 49, 50, 51, or 52; and (viii) the eighth first target nucleic acid contains SEQ ID NO: 53, 54, 55, 56, 57, 58, 59, 60, 61, 62, 63, 64, 65, 66, 67, 68, 69, 70, 71, 72, 73, or 74. For the oligo-nucleotides, (i) the oligo-nucleotides in the first pair of primers are, SEQ ID NOs: 75 and 76, respectively; SEQ ID NOs: 77 and 78, respectively; or SEQ ID NOs: 79 and 80, respectively; (ii) the oligo-nucleotides in the second pair of primers are, SEQ ID NOs: 81 and 82, respectively; SEQ ID NOs: 81 and 177, respectively; SEQ ID NOs: 83 and 84, respectively; SEQ ID NOs: 83 and 178, respectively; SEQ ID NOs: 85 and 86, respectively; SEQ ID NOs: 87 and 88, respectively; SEQ ID NOs: 89 and 90, respectively; SEQ ID NOs: 89 and 181, respectively; SEQ ID NOs: 91 and 92, respectively; SEQ ID NOs: 93 and 94, respectively; SEQ ID NOs: 179 and 180, respectively; SEQ ID NOs: 182 and 183, respectively; or SEQ ID NOs: 184 and 185, respectively; (iii) the oligo-nucleotides in the third pair of primers are, SEQ ID NOs: 95 and 96, respectively; (iv) the oligo-nucleotides in the fourth pair of primers are, SEQ ID NOs: 97 and 98, respectively; SEQ ID NOs: 186 and 98, respectively; SEQ ID NOs: 99 and 100, respectively; SEQ ID NOs: 99 and 187, respectively; SEQ ID NOs: 101 and 102, respectively; SEQ ID NOs: 101 and 188, respectively; SEQ ID NOs: 103 and 104, respectively; SEQ ID NOs: 103 and 189, respectively; SEQ ID NOs: 105 and 106, respectively; SEQ ID NOs: 105 and 190, respectively; SEQ ID NOs: 107 and 108, respectively; SEQ ID NOs: 191 and 108, respectively; SEQ ID NOs: 109 and 110, respectively; or SEQ ID NOs: 192 and 110, respectively; (v) the oligo-nucleotides in the fifth pair of primers are, SEQ ID NOs: 111 and 112, respectively; SEQ ID NOs: 113 and 114, respectively; SEQ ID NOs: 115 and 116, respectively; SEQ ID NOs: 115 and 195, respectively; SEQ ID NOs: 117 and 118, respectively; SEQ ID NOs: 119 and 120, respectively; SEQ ID NOs: 121 and 122, respectively; SEQ ID NOs: 123 and 124, respectively; or SEQ ID NOs: 193 and 194 respectively; (vi) the oligo-nucleotides in the sixth pair of primers are, SEQ ID NOs: 125 and 126, respectively; SEQ ID NOs: 125 and 196, respectively; SEQ ID NOs: 127 and 128, respectively; SEQ ID NOs: 197 and 128, respectively; SEQ ID NOs: 129 and 130, respectively; SEQ ID NOs: 129 and 198, respectively; SEQ ID NOs: 131 and 132, respectively; or SEQ ID NOs: 199 and 200, respectively; (vii) the oligo-nucleotides in the seventh pair of primers are, SEQ ID NOs: 133 and 134, SEQ ID NOs: 201 and 134, respectively; SEQ ID NOs: 135 and 136, respectively; SEQ ID NOs: 202 and 136, respectively; SEQ ID NOs: 137 and 138, respectively; SEQ ID NOs: 203 and 138, respectively; SEQ ID NOs: 139 and 140, respectively; SEQ ID NOs: 204 and 140, respectively; SEQ ID NOs: 141 and 142, respectively; SEQ ID NOs: 205 and 142, respectively; SEQ ID NOs: 143 and 144, respectively; SEQ ID NOs: 206 and 144, respectively; SEQ ID NOs: 145 and 146, respectively; SEQ ID NOs: 145 and 207, respectively; SEQ ID NOs: 147 and 148, respectively; or SEQ ID NOs: 208 and 148, respectively; and (viii) the oligo-nucleotides in the eighth pair of primers are, SEQ ID NOs: 149 and 150, respectively; SEQ ID NOs: 151 and 152, respectively; SEQ ID NOs: 153 and 154, respectively; SEQ ID NOs: 155 and 156, respectively; SEQ ID NOs: 157 and 158, respectively; SEQ ID NOs: 217 and 158, respectively; SEQ ID NOs: 159 and 160, respectively; SEQ ID NOs: 159 and 218, respectively; SEQ ID NOs: 161 and 162, respectively; SEQ ID NOs: 163 and 164, respectively; SEQ ID NOs:163 and 227, respectively; SEQ ID NOs: 165 and 166, respectively; SEQ ID NOs: 167 and 168, respectively; SEQ ID NOs: 169 and 170, respectively; SEQ ID NOs: 171 and 172, respectively; SEQ ID NOs: 173 and 174, respectively; SEQ ID NOs: 175 and 176, respectively; SEQ ID NOs: 209 and 210, respectively; SEQ ID NOs: 211 and 212, respectively; SEQ ID NOs: 213 and 214, respectively; SEQ ID NOs: 215 and 216, respectively; SEQ ID NOs: 219 and 220, respectively; SEQ ID NOs: 221 and 222, respectively; SEQ ID NOs: 223 and 224, respectively; SEQ ID NOs: 225 and 226, respectively; SEQ ID NOs: 228 and 229 respectively; or SEQ ID NOs: 230 and 176, respectively.

In an preferred embodiment of the set of target nucleic acids of this invention, the first target nucleic acid contains SEQ ID NO: 1, 2, or 3; (b) the second target nucleic acid contains SEQ ID NO: 7, 8, 9, or 10; (c) the third target nucleic acid contains SEQ ID NO: 14 or 15; (d) the fourth target nucleic acid contains SEQ ID NO: 23, 24, 25, 26, 27, or 28; (e) the fifth target nucleic acid contains SEQ ID NO: 31, 32, 33, or 34; (f) the sixth target nucleic acid contains SEQ ID NO: 41, 42, or 43; (g) the seventh target nucleic acid contains SEQ ID NO: 45, 46, or 47; and (h) the eighth target nucleic acid contains SEQ ID NO: 53, 55, 56, or 57. For the oligo-nucleotides, (a) the oligo-nucleotides in the second pair of primers are, respectively, SEQ ID NOs: 81 and 82, SEQ ID NOs: 81 and 177, SEQ ID NOs: 83 and 84, SEQ ID NOs: 83 and 178, SEQ ID NOs: 85 and 86, SEQ ID NOs: 87 and 88, or SEQ ID NOs: 179 and 180; (b) the oligo-nucleotides in the fourth pair of primers are, respectively, SEQ ID NOs: 97 and 98, SEQ ID NOs: 186 and 98, SEQ ID NOs: 99 and 100, SEQ ID NOs: 99 and 187, SEQ ID NOs: 101 and 102, SEQ ID NOs: 101 and 188, SEQ ID NOs: 103 and 104, SEQ ID NOs: 103 and 189, SEQ ID NOs: 105 and 106, or SEQ ID NOs: 105 and 190; (c) the oligo-nucleotides in the fifth pair of primers are, respectively, SEQ ID NOs: 111 and 112, SEQ ID NOs: 113 and 114, SEQ ID NOs: 115 and 116, SEQ ID NOs: 115 and 195, SEQ ID NOs: 117 and 118, or SEQ ID NOs: 193 and 194; (d) the oligo-nucleotides in the sixth pair of primers are, respectively, SEQ ID NOs: 125 and 126, SEQ ID NOs: 125 and 196, SEQ ID NOs: 127 and 128, SEQ ID NOs: 197 and 128, SEQ ID NOs: 129 and 130, or SEQ ID NOs: 129 and 198; (e) the oligo-nucleotides in the seventh pair of primers are, respectively, SEQ ID NOs: 133 and 134, SEQ ID NOs: 201 and 134, SEQ ID NOs: 135 and 136, SEQ ID NOs: 202 and 136, SEQ ID NOs: 137 and 138, or SEQ ID NOs: 203 and 138; and (f) the oligo-nucleotides in the eighth pair of primers are, respectively, SEQ ID NOs: 149 and 150, SEQ ID NOs: 151 and 152, SEQ ID NOs: 153 and 154, SEQ ID NOs: 209 and 210, SEQ ID NOs: 211 and 212, or SEQ ID NOs: 213 and 214.

Another aspect of this invention features a set of nucleic acids comprising one or more of the above-described first, second, third, fourth, fifth, sixth, seventh, and eighth pairs of primers.

A further aspect of this invention features a method of evaluating responsiveness of a subject to a drug. The method includes (1) providing a nucleic acid sample from a subject; (ii) determining a single nucleotide polymorphism genotype (SNP genotype) of a gene group that contains one or more of the human ADAR, CASP5, FGF1, ICSBP1, IFI44, PIK3CG, TAP2, and TGFBRAP1 genes; and (iii) comparing the single nucleotide polymorphism genotype with a predetermined SNP genotype. A subject is predicted to be responsive or non-responsive to the drug if the SNP genotype is identical to a predetermined SNP genotype.

A “genotype” refers to a specific allelic composition of an organism, a genome, or a part of a genome. In particular, this term refers to alleles of a particular gene or set of genes. A single nucleotide polymorphism or SNP genotype refers to the SNP composition of a gene or set of genes. It is presented as a string of nucleotides. For example, a region of human FGF1 gene on each chromosome contains two SNP sites (VGV567 and VGV679). If the two chromosomes of a subject have (1) C and T at the VGV567 sites and (2) A and A at the VGV567 sites, the FGF VGV567/VGV679 SNP genotype of this subject is represented as “CTAA.”

In one embodiment, the aforementioned nucleic acid sample contains one or more of the above-described SNP-containing target nucleic acids, which can be obtained from amplification of the corresponding human gene nucleic acid template with a pair of primers. The method can be used to evaluate drug responsiveness of a subject that has or is suspected of having a liver disorder, such as hepatitis (e.g., hepatitis C), liver fibrosis, or liver cirrhosis. Examples of a drug include type I interferon (e.g., interferon-alpha and interferon-beta), ribavirin, or their combination. In one example, a subject is predicted to be responsive or non-responsive to type I interferon if his or her SNP genotype is identical to a predetermined SNP genotype such as one of those reference genotypes listed in Table 4 in Example 1 below. A reference genotype refers to a SNP genotype known to be associated with the responsiveness or non-responsiveness to a certain drug. It can be determined in the manner described in Example 1 below.

The details of one or more embodiments of the invention are set forth in the accompanying description below. Other advantages, features, and objects of the invention will be apparent from the detailed description and the claims.

### DETAILED DESCRIPTION

This invention relates to nucleic acids and a method of using the nuclei acids for predicting a subject's responsiveness to a drug based on an SNP genotype of the subject. The method requires determining a subject's SNP genotype of a gene or a set of genes and predicting the subject's responsiveness to the drug by comparing the SNP genotype with reference genotypes known to be associated with responsiveness or non-responsiveness to the drug.

One can select a gene or set of genes that encode proteins involved in the metabolism or signal transduction pathway of the drug of interest. For example, interferon (IFN) or ribavirin is often used in treating hepatitis C. Genes encoding proteins involved in IFN signaling pathways or immunomodulation can be selected for SNP genotyping. As demonstrated in Example 1 below, the SNP genotypes of a set of certain genes showed statistically significant differences between subjects responsive to IFN/ribavirin and subjects non-responsive to IFN/ribavirin. To predict a subject's responsiveness to IFN/ribavirin, one can determine the subject's corresponding SNP genotype of one or more of these genes.

Many technologies known in the art can be used to genotype SNPs (see, e.g., Kwok, Pharmacogenomics, 2000, vol 1, pp 95-100. “High-throughput genotyping assay approaches”). These technologies are based on direct sequencing, allele specific oligonucleotide hybridization, oligonucleotide elongation by dideoxynucleotides or ddNTPs optionally in the presence of deoxynucleotides, ligation of allele specific oligonucleotides, or cleavage of allele specific oligonucleotides. Each of these technologies can be coupled to a detection system such as measurement of direct or polarized fluorescence, or mass spectrometry.

The genotyping assays can be carried out on a product obtained from amplification of the DNA of a subject. This product and corresponding primers are selected to cover a polynucleotide region containing a SNP of interest. Amplification techniques known in the art include, but are not limited to, cloning, polymerase chain reaction (PCR), polymerase chain reaction of specific alleles (ASA), ligase chain reaction (LCR), nested polymerase chain reaction, self sustained sequence replication (Guatelli, et al., 1990, Proc. Natl. Acad. Sci. USA 87:1874-1878), transcriptional amplification system (Kwoh, et al., 1989, Proc. Natl. Acad. Sci. USA 86:1173-1177), and Q-Beta Replicase (Lizardi, et al., 1988, Bio/Technology 6:1197).

Amplification products can be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, primer extension, sequencing, hybridization, and the like. PCR based amplification techniques include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection techniques, e.g., microarraies, allow the differential detection of multiple PCR products in a sample simultaneously.

After determining a subject's SNP genotype, one can compare it with a reference genotype known to be associated with responsiveness or non-responsiveness to a drug. If the SNP genotype is identical to a reference genotype associated with responsiveness (or non-responsiveness), the subject is predicted to be responsive (or non-responsive). When comparing with reference SNP genotypes of more than one gene, one might obtain contradictory conclusions. In this case, one should take into account of the weight, i.e., prediction power, of each gene. Table 4 in Example 1 below lists exemplary reference SNP genotypes of 8 genes and corresponding weights.

For high throughput comparison, one can incorporate reference SNP genotypes of all genes of interest and the corresponding weights into a logical formula, which has a number of variables corresponding to the genes, respectively. After substituting a subject's SNP genotype of each gene for each variable, one can obtain a predicting result returned from the formula. An exemplary logical formula is shown below:

$$p = \frac{exp ( \sum ( gene \times weight ) )}{1 + exp ( \sum ( gene \times weight ) )}$$

In the formula, “p” stands for the probability of a subject to be responsive to a treatment. “Gene” is a value (e.g., +1, −1, or 0) for a reference SNP genotype of a gene that is associated with the responsiveness or non-responsiveness to a drug. “Weight” represents” the weight/prediction power of each gene as mentioned above.

One can obtain reference SNP genotypes in a manner similar to that described in Example 1 below. More specifically, one can (1) select a sample group of subjects who are known to be responsive or non-responsive to a drug of interest, (2) SNP type a set of candidate genes, and (3) identify the degree of association between an SNP genotype and a responding status using statistic methods known in the art. The genotyping data is then analyzed to estimate the distribution frequency of the different alleles observed in the studied sample. Calculation of the allelic frequencies can be carried out with the assistance of a software program such as SAS® statistical package version 8, SAS-suite® (SAS) or SPLUS® (MathSoft). Comparison of the allelic distributions of a SNP across different ethnic groups can also be carried out by means of the software ARLEQUIN® and SAS-suite®.

The above-described methods and nucleic acids can be used to predict a hepatitis C patient's responsiveness to IFN. They can also be used in predicting IFN responsiveness of a subject that has or is suspected of having disorders that can be treated by IFN. Examples of these disorders include (1) diseases caused by viruses, such as hepatitis (A, B, C, D, E, F, G types) virus, HIV, influenza virus, herpes virus, adenovirus, human polyomavirus, human papilloma virus, human parbovirus, Mumps virus, human rotavirus, enterovirus, Japanese B Encephalitis virus, dengue virus, rubella virus, and HTLV; (2) diseases caused by bacteria, such as Staphylococcus aureus, hemolytic streptococcus, pathogenic Escherichia coli, enteritis vibrio, Helicobacter pylori, Campylobacter, Vibrio cholerae, dysentery bacilli, salmonellae, Yelsinia, Neisseria gonorrhoeae, Listeria, Leptospira, Legionella, spirochete, Mycoplasma pneumoniae, rickettsiae, chlamydiae, malaria plasmodia, dysentery amoeba, and pathogenic fungi; (3) diseases caused by parasites and Eumycetes, and (4) oncological diseases, such as retinoblastoma, Wilm's tumor, familial colonic polyposis, hereditary non polyposis colon cancer, neurofibromatosis, familial chest cancer, xeroderma pigmentosum, blain cancer, oral cancer, esophageal cancer, stomach cancer, colon cancer, liver cancer, pancreatic cancer, lung cancer, thyroid cancer, mammary gland tumor, urinary tumor, virilia tumor, muliebria tumor, skin tumor, osteosarcoma, osteochondrosarcoma, leukemia, lymphoma, and solid tumor.

In addition, as shown in Example 2 below, SNPs of 8 genes were used to accurately predict patients' responsiveness to drugs. The results indicate that the 8 genes (i) are associated with the drug responsiveness and (ii) play important roles in the metabolism or signal transduction pathways of a drug, such as IFN or ribavirin. Accordingly, these genes and SNPs can be used as targets for drug development and as genetic markers for molecular diagnosis, respectively. Indeed, a compound that (i) binds to a protein encoded by one of the genes or (ii) regulates the expression level or activity of the protein is a drug candidate for treating one of the above-mentioned disorders, e.g., a viral infection, such as hepatitis C. Thus, within the scope of this invention is a method for identifying a compound or composition for treating one of the above-mentioned disorders, such as an infection with a virus, e.g., hepatitis virus. The method includes (1) obtaining a first polypeptide that contains a sequence encoded by one of the aforementioned 8 genes; (2) contacting a compound with the first polypeptide; and (3) detecting a binding between the first polypeptide and the compound. The compound is determined to be effective in treating the disorder if the compound binds to the first polypeptide, but not to a second polypeptide that is identical to the first polypeptide, except that the second polypeptide does not have the sequence encoded by one of the aforementioned 8 genes. Also within the scope of this invention is a method for identifying a compound for treating one of the above-mentioned disorders, e.g., a viral infection The method includes (1) obtaining a first system, e.g., a cell, containing or capable of expressing a polypeptide encoded by one of the 8 genes; (2) incubating the first system in a medium containing a compound; and (3) determining an expression or activity level of the polypeptide. The compound is determined to be effective in treating the disorder if the expression or activity level differs from that determined in the same manner from a second system except that the second system is incubated in a medium free of the compound.

The two specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

In this example, genetic markers related to IFN signaling pathways or immunomodulation were analyzed for negative or positive association with IFN/ribavirin treatment for chronic hepatitis C(CHC).

### Subjects

Blood samples were collected from 221 chronic hepatitis C patients at National Taiwan University Hospital, Kaohsiung Medical University Hospital, and Tri-Service General Hospital in Taiwan. Informed consent and medical record history were obtained from each patient. All of the patients had received IFN-α (3-5 MU 3 time/per week) and ribavirin (1200 mg/per day) combination therapy for 6-12 months and were followed up for at least 6 months. Patients that showed HCV RNA (+) to HCV RNA (−) conversion 6 months after treatment were defined as “sustained responders (SR)” to this combination treatment. Those still remained HCV RNA (+) were defined as “non-responders (NR).”

### Genotyping

Genomic DNAs were extracted from each of the blood samples by standard techniques. The quality of the extracted genomic DNAs was checked by agarose gel electrophoresis analysis and stored at −80° C. until use. More than 1,500 SNPs in 150 candidate genes were SNP genotyped.

SNP genotyping was conducted by DNA sequencing. More specifically, fragments of candidate genes were amplified by the PCR reaction. The reaction mixture contained Tris-HCl 100 mM (pH 8.3), KCl 50 mM, MgCl₂ 2.5 mM, 20 ng genomic DNA, 0.2 mM dNTP mixture, each of 0.2 μM forward and reversed primers, 5 U of VioTaq DNA polymerase (VIOGENE) and 0.025 U of Pfu DNA polymerase (Stratagene) in a total volume of 50 μL reaction. The PCR was performed using a touch-down program including an initial denaturing at (i) 94° C. for 4 minutes; (ii) 10 cycles of: melting at 94° C. for 40 seconds, annealing at 70° C. with 1° C. decrement per cycle for 40 seconds, and extending at 72° C. for 1 minute 30 seconds; (iii) 25 cycles of: melting at 94° C. for 40 seconds, annealing at 60° C. for 40 seconds, and extending at 72° C. for 1 minute 30 seconds; and (iv) one cycle of final extension at 72° C. for 10 minutes. The amplification was carried out on 2700 PCR machines (ABI). The amplified products were purified by membrane ultra-filtration with a MultiScreen PCR plate (Millipore) according to the manufacture's instructions. PCR products from each gene were then sequenced by a sequencer. Each sequencing reaction mixture contained corresponding PCR products, Big Dye Terminator Ready-Reaction-Premix, and 10 pmol of a sequencing primer. The sequencing was performed using a program of 28 cycles at 94° C. for 30 seconds, 48° C. for 30 seconds, and 58° C. for 2 minutes. After the sequencing, the reaction products were loaded on an ABI 3700 capillary sequencer. DNA sequence date and track were collected by ABI DNA Sequence Analyzer. Phrad was used to perform base calling and sequencing assembling. The initial identification of potential SNP sites was performed by Polyphred (Department of Genome Sciences, University of Washington, Seattle, Wash.). PolyPhred is a software program that compares fluorescence-based sequences across traces obtained from different individuals to identify heterozygous sites for single nucleotide substitutions. Its functions are integrated with the use of three other programs also from the same source: Phred (a base-caller program), Phrap (an assembler program), and Consed (a Unix-based graphical editor and automated finishing program for Phrap Sequence Assemblies). PolyPhred identifies potential heterozygotes using the base calls and peak information provided by Phred and the sequence alignments provided by Phrap. Potential heterozygotes identified by PolyPhred are marked for rapid inspection using the Consed tool. Finally, the genotype of each tested individual was determined by computer software and confirmed manually.

Alternatively, SNP genotyping was conducted by the Template-directed Dye Terminator Incorporation assay with Fluorescent Polarization detection (FP-TDI, Washington University School of Medicine, St. Louis, Mich.). Primers were designed for each SNP site of the candidate genes. The SNP primers used for genotyping SNPs in the 8 genes described above are listed in Table 2. For each typing, PCR was performed in a total volume of 10 μl of 1×PCR buffer (Applied Biosystems), 2.5 mM MgCl₂, 50 M dNTP, 0.1 M each of forward and reverse primers, and 0.2 U AmpliTaq Gold DNA polymerase. The thermal cycle condition for the PCR reaction was: activation step at 94° C. for 10 minutes, 35 cycles at 95° C. for 10 seconds, 55° C. for 20 seconds, and 72° C. for 30 seconds, ending with 10 minutes at 72° C. After the PCR reaction, excessive PCR primers and dNTPs were cleansed in a degradation reaction. A PCR clean-up reagent for this degradation reaction contained 1 U shrimp alkaline phosphatase and 1 U E. coli exonuclease I in a shrimp alkaline phosphatase buffer (25 mM Tris-HCl, pH 8.5 and 2.5 mM MgCl2). The reaction was performed at 37° C. for 1 hour, followed by an incubation at 80° C. for 15 minutes. Single-base extension was then performed in a solution containing 1× reaction buffer (Perkin Elmer), 0.25 M SNP primer, 1 μl Acyclo Terminator Mix (Perkin Elmer), and 0.25 U AcycloPol DNA polymerase (Perkin Elmer) under the following conditions: 95° C. for 2 minutes, 25 to 50 cycles of 95° C. for 15 seconds, and 55° C. for 30 seconds. Then, fluorescence-polarization was measured on a Perkin Elmer fluorescence reader to detect the distribution of SNPs of 8 genes.

### Estimation of SNP Haplotype and Frequencies

In this study, a computer program PHASE was used to estimate haplotypes and their frequencies on the basis of unphased genotype data. A Bayesian algorithm was used with this program PHASE to examine the unknown haplotypes as unobserved random quantities for evaluating their frequencies and the conditional distribution of multilocus haplotypes in diploid populations. The D′ and r² measures were used to analyze linkage disequilibrium between pairs of SNPs.

### Statistical Analysis

Statistical analyses were performed using the SAS statistical package version 8®. The χ² or Fisher exact test was used to compare the genotypes and allele frequencies between SR and NR of each SNP. The χ² or Fisher exact test was also performed to evaluate the combined genotypes of multiple SNPs in the same gene or pathway. Multiple-logistic regression was performed to evaluate whether there was a difference in response for each SNP after adjustment for age, gender, living habits, and related viral factors. All statistical tests were 2-tailed and P-values less than 0.05 were considered statistically significant.

### Results

It was shown that a collection of SNPs of 8 genes, i.e., ADAR, CASP5, FGF1, ICSBP1, IFI44, PIK3CG, TAP2, and TGFBRAP1, were strongly associated with the responsiveness in hepatitis C patients. These 8 genes were selected by association analysis of responsive status using major haplotype frequencies from each gene in the candidate gene pool. A set of SNPs of the 8 genes that retained most of the information available in the full haplotype (htSNP) of each gene were selected and listed in Table 3.

TABLE 3
Selected Sets of SNPs

| Gene | SNP ID |
|---|---|
| ADAR | VGV1473, VGV1600, VGV1798 |
| CASP5 | rs518604, rs2282658, rs484345, rs1699087 |
| FGF1 | VGV567, VGV679 |
| ICSBP1 | rs385989, rs305067, VGV1824, VGV1826, |
| | rs305088, VGV1827 |
| IFI44 | VGV199, VGV2188, VGV2191, VGV33 |
| PIK3CG | rs1526083, rs3779501, rs2037718 |
| TAP2 | rs1871665, rs2071543, rs1800453 |
| TGFBRAP1 | VGV2200, VGV2204, VGV2197, VGV2325 |

Then, the SNP genotypes associated with responsiveness to the IFN-α/ribavirin treatment or non-responsiveness to the treatment were obtained. The SNP genotypes that were associated with responsiveness (“R”) or non-responsiveness (“NR”), i.e., reference genotypes, were summarized in Table 4 below. Also included in Table 4 was the weight of each gene. Based on the weight, the prediction power, i.e., priority, of each gene was also determined and shown in Table 4

TABLE 4
Reference Genotypes and Weights

| | | | | |
|---|---|---|---|---|
| Priority | GENE | Reference Genotype | Responsiveness | Weight |

| | | | | |
|---|---|---|---|---|
| 1 | TGFBRAP1 | CCCCCTTT | R | 11.7689 |
| | | CCCCTTTT | R | |
| | | CCCTCTCT | R | |
| | | CCTTCCCC | R | |
| 5 | PIK3CG | AACTGG | NR | 2.5411 |
| 8 | ICSBP1 | GGCCGGCGTTCC | R | 0.122 |
| | | GTGGGGAACCGG | R | |
| | | TTCCGGAATCGA | R | |
| | | TTCCGGAATCGG | R | |
| | | TTCCGGAATTGG | R | |
| | | TTCGGGAACCGG | R | |
| | | TTCGGGAATCGG | R | |
| | | TTGGGGAACCGG | R | |
| 3 | IF144 | GGTTAAAG | R | 2.937 |
| | | GGTTAGGG | NR | |
| | | GTTTAAAA | R | |
| | | GTTTAAAG | R | |
| | | GTTTAGGG | NR | |
| 4 | TAP2 | CACTCC | R | 2.6099 |
| 2 | ADAR | CCAACC | R | 2.9426 |
| | | CTAGCC | NR | |
| | | TTGGCC | NR | |
| | | TTGGCT | NR | |
| 6 | CASP5 | AACCAACA | NR | 1.5063 |
| | | AACGGACA | NR | |
| | | GACCAACA | NR | |
| | | GACCAACC | NR | |
| | | GACCGACA | R | |
| | | GACCGGAA | R | |
| | | GACGGACA | R | |
| | | GACGGGAA | R | |
| 7 | FGF1 | CTAA | R | 1.1484 |

### EXAMPLE 2

The just-described set of SNPs of the 8 genes was used to predict the responsiveness of 221 patients to IFN α/ribavirin treatment in a blind test. The SNP genotypes of the 221 patients were determined by the method described above. Different numbers of patients were selected randomly for each gene. The responsiveness to IFN α/ribavirin treatment were predicted based on the reference SNP genotypes of ADAR, CASP5, FGF1, ICSBP1, IFI44, PIK3CG, TAP2, and TGFBRAP1, respectively. The results are shown in Table 5 below.

TABLE 5
Predicting Responsiveness to IFN-α/ribavirin

| | Patients | Correctly | | |
|---|---|---|---|---|
| Gene | Included | Predicted | Accuracy | Coverage |
| ADAR | 60 | 48 | 80.00% | 27.15% |
| CASP5 | 54 | 41 | 75.90% | 24.43% |
| FGF1 | 102 | 67 | 65.70% | 46.15% |
| ICSBP1 | 81 | 64 | 79.00% | 36.65% |
| IFI44 | 62 | 49 | 79.00% | 28.05% |
| PIK3CG | 15 | 11 | 73.30% | 6.78% |
| TAP2 | 9 | 6 | 66.70% | 4.07% |
| TGFBRAP1 | 38 | 31 | 81.60% | 17.19% |

The results show that the prediction is highly accurate. The results also indicate that SNPs of the 8 genes are associated with the drug responsiveness and the genes indeed play important roles in the metabolism or signal transduction pathways of an anti-viral drug, such as IFN or ribavirin. Accordingly, the genes and the corresponding SNPs can be used as targets for anti-viral drug development and as genetic markers for molecular diagnosis, respectively.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A set of target nucleic acids comprising one or more of
a first target nucleic acid obtained from amplification of the human ADAR gene nucleic acid template with a first pair of primers, each containing an oligo-nucleotide selected from the ADAR gene region;
a second target nucleic acid obtained from amplification of the human CASP5 gene nucleic acid template with a second pair of primers, each containing an oligo-nucleotide selected from the CASP5 gene region;
a third target nucleic acid obtained from amplification of the human FGF1 gene nucleic acid template with a third pair of primers, each containing an oligo-nucleotide selected from the FGF1 gene region;
a fourth target nucleic acid obtained from amplification of the human ICSBP1 gene nucleic acid template with a fourth pair of primers, each containing an oligo-nucleotide selected from the ICSBP1 gene region;
a fifth target nucleic acid obtained from amplification of the human IFI44 gene nucleic acid template with a fifth pair of primers, each containing an oligo-nucleotide selected from the IFI44 gene region;
a sixth target nucleic acid obtained from amplification of the human PIK3CG gene nucleic acid template with a sixth pair of primers, each containing an oligo-nucleotide selected from the PIK3CG gene region;
a seventh target nucleic acid obtained from amplification of the human TAP2 gene nucleic acid template with a seventh pair of primers, each containing an oligo-nucleotide selected from the TAP2 gene region; and
an eighth target nucleic acid obtained from amplification of the human TGFBRAP1 gene nucleic acid template with a eighth pair of primers, each containing an oligo-nucleotide selected from the TGFBRAP1 gene region;
wherein each target nucleic acid has a nucleotide at a single nucleotide polymorphism site and is 20-1,000 nucleotides in length.

2. The set of target nucleic acids of claim 1, wherein each oligo-nucleotide is 14-50 nucleotides in length.

3. The set of target nucleic acids of claim 2, wherein each oligo-nucleotide is 14-40 nucleotides in length.

4. The set of target nucleic acids of claim 1, wherein each target nucleic acid is 200-1,000 nucleotides in length.

5. The set of target nucleic acids of claim 1, wherein
the oligo-nucleotides in the first pair of primers are, respectively, SEQ ID NOs: 75 and 76, SEQ ID NOs: 77 and 78, or SEQ ID NOs: 79 and 80;
the oligo-nucleotides in the second pair of primers are, respectively, SEQ ID NOs: 81 and 82, SEQ ID NOs: 81 and 177, SEQ ID NOs: 83 and 84, SEQ ID NOs: 83 and 178, SEQ ID NOs: 85 and 86, SEQ ID NOs: 87 and 88, SEQ ID NOs: 89 and 90, SEQ ID NOs: 89 and 181, SEQ ID NOs: 91 and 92, SEQ ID NOs: 93 and 94, SEQ ID NOs: 179 and 180, SEQ ID NOs: 182 and 183, or SEQ ID NOs: 184 and 185;
the oligo-nucleotides in the third pair of primers are, respectively, SEQ ID NOs: 95 and 96;
the oligo-nucleotides in the fourth pair of primers are, respectively, SEQ ID NOs: 97 and 98, SEQ ID NOs: 186 and 98, SEQ ID NOs: 99 and 100, SEQ ID NOs: 99 and 187, SEQ ID NOs: 101 and 102, SEQ ID NOs: 101 and 188, SEQ ID NOs: 103 and 104, SEQ ID NOs: 103 and 189, SEQ ID NOs: 105 and 106, SEQ ID NOs: 105 and 190, SEQ ID NOs: 107 and 108, SEQ ID NOs: 191 and 108, SEQ ID NOs: 109 and 110, or SEQ ID NOs: 192 and 110;
the oligo-nucleotides in the fifth pair of primers are, respectively, SEQ ID NOs: 111 and 112, SEQ ID NOs: 113 and 114, SEQ ID NOs: 115 and 116, SEQ ID NOs: 115 and 195, SEQ ID NOs: 117 and 118, SEQ ID NOs: 119 and 120, SEQ ID NOs: 121 and 122, SEQ ID NOs: 123 and 124, or SEQ ID NOs: 193 and 194;
the oligo-nucleotides in the sixth pair of primers are, respectively, SEQ ID NOs: 125 and 126, SEQ ID NOs: 125 and 196, SEQ ID NOs: 127 and 128, SEQ ID NOs: 197 and 128, SEQ ID NOs: 129 and 130, SEQ ID NOs: 129 and 198, SEQ ID NOs: 131 and 132, or SEQ ID NOs: 199 and 200;
the oligo-nucleotides in the seventh pair of primers are, respectively, SEQ ID NOs: 133 and 134, SEQ ID NOs: 201 and 134, SEQ ID NOs: 135 and 136, SEQ ID NOs: 202 and 136, SEQ ID NOs: 137 and 138, SEQ ID NOs: 203 and 138, SEQ ID NOs: 139 and 140, SEQ ID NOs: 204 and 140, SEQ ID NOs: 141 and 142, SEQ ID NOs: 205 and 142, SEQ ID NOs: 143 and 144, SEQ ID NOs: 206 and 144, SEQ ID NOs: 145 and 146, SEQ ID NOs: 145 and 207, SEQ ID NOs: 147 and 148, or SEQ ID NOs: 208 and 148; and
the oligo-nucleotides in the eighth pair of primers are, respectively, SEQ ID NOs: 149 and 150, SEQ ID NOs: 151 and 152, SEQ ID NOs: 153 and 154, SEQ ID NOs: 155 and 156, SEQ ID NOs: 157 and 158, SEQ ID NOs: 217 and 158, SEQ ID NOs: 159 and 160, SEQ ID NOs: 159 and 218, SEQ ID NOs: 161 and 162, SEQ ID NOs: 163 and 164, SEQ ID NOs:163 and 227, SEQ ID NOs: 165 and 166, SEQ ID NOs: 167 and 168, SEQ ID NOs: 169 and 170, SEQ ID NOs: 171 and 172, SEQ ID NOs: 173 and 174, SEQ ID NOs: 175 and 176, SEQ ID NOs: 209 and 210, SEQ ID NOs: 211 and 212, SEQ ID NOs: 213 and 214, SEQ ID NOs: 215 and 216, SEQ ID NOs: 219 and 220, SEQ ID NOs: 221 and 222, SEQ ID NOs: 223 and 224, SEQ ID NOs: 225 and 226, SEQ ID NOs: 228 and 229, or SEQ ID NOs: 230 and 176.

6. The set of target nucleic acids of claim 5, wherein each oligo-nucleotide is 14-50 nucleotides in length.

7. The set of target nucleic acids of claim 6, wherein each oligo-nucleotide is 14-40 nucleotides in length.

8. The set of target nucleic acids of claim 5, wherein
the oligo-nucleotides in the second pair of primers are, respectively, SEQ ID NOs: 81 and 82, SEQ ID NOs: 81 and 177, SEQ ID NOs: 83 and 84, SEQ ID NOs: 83 and 178, SEQ ID NOs: 85 and 86, SEQ ID NOs: 87 and 88, or SEQ ID NOs: 179 and 180;
the oligo-nucleotides in the fourth pair of primers are, respectively, SEQ ID NOs: 97 and 98, SEQ ID NOs: 186 and 98, SEQ ID NOs: 99 and 100, SEQ ID NOs: 99 and 187, SEQ ID NOs: 101 and 102, SEQ ID NOs: 101 and 188, SEQ ID NOs: 103 and 104, SEQ ID NOs: 103 and 189, SEQ ID NOs: 105 and 106, or SEQ ID NOs: 105 and 190;
the oligo-nucleotides in the fifth pair of primers are, respectively, SEQ ID NOs: 111 and 112, SEQ ID NOs: 113 and 114, SEQ ID NOs: 115 and 116, SEQ ID NOs:115 and 195, SEQ ID NOs: 117 and 118, or SEQ ID NOs: 193 and 194;
the oligo-nucleotides in the sixth pair of primers are, respectively, SEQ ID NOs: 125 and 126, SEQ ID NOs: 125 and 196, SEQ ID NOs: 127 and 128, SEQ ID NOs: 197 and 128, SEQ ID NOs: 129 and 130, or SEQ ID NOs: 129 and 198;
the oligo-nucleotides in the seventh pair of primers are, respectively, SEQ ID NOs: 133 and 134, SEQ ID NOs: 201 and 134, SEQ ID NOs: 135 and 136, SEQ ID NOs: 202 and 136, SEQ ID NOs: 137 and 138, or SEQ ID NOs: 203 and 138; and
the oligo-nucleotides in the eighth pair of primers are, respectively, SEQ ID NOs: 149 and 150, SEQ ID NOs: 151 and 152, SEQ ID NOs: 153 and 154, SEQ ID NOs: 209 and 210, SEQ ID NOs: 211 and 212, or SEQ ID NOs: 213 and 214.

9. The set of target nucleic acids of claim 1, wherein
the first target nucleic acid contains SEQ ID NO: 1, 2, 3, 4, 5, or 6;
the second target nucleic acid contains SEQ ID NO: 7, 8, 9, 10, 11, 12, or 13;
the third target nucleic acid contains SEQ ID NO: 14, 15, 16, 17, 18, 19, 20, 21, or 22;
the fourth target nucleic acid contains SEQ ID NO: 23, 24, 25, 26, 27, 28, 29, or 30;
the fifth target nucleic acid contains SEQ ID NO: 31, 32, 33, 34, 35, 36, 37, 38, 39, or 40;
the sixth target nucleic acid contains SEQ ID NO: 41, 42, 43, or 44;
the seventh target nucleic acid contains SEQ ID NO: 45, 46, 47, 48, 49, 50, 51, or 52; and
the eighth first target nucleic acid contains SEQ ID NO: 53, 54, 55, 56, 57, 58, 59, 60, 61, 62, 63, 64, 65, 66, 67, 68, 69, 70, 71, 72, 73, or 74.

10. The set of target nucleic acids of claim 9, wherein each target nucleic acid is 200-1,000 nucleotides in length.

11. The set of target nucleic acids of claim 9, wherein each oligo-nucleotide is 14-50 nucleotides in length.

12. The set of target nucleic acids of claim 11, wherein each oligo-nucleotide is 14-40 nucleotides in length.

13. The set of target nucleic acids of claim 9, wherein
the first target nucleic acid contains SEQ ID NO: 1, 2, or 3;
the second target nucleic acid contains SEQ ID NO: 7, 8, 9, or 10;
the third target nucleic acid contains SEQ ID NO: 14 or 15;
the fourth target nucleic acid contains SEQ ID NO: 23, 24, 25, 26, 27, or 28;
the fifth target nucleic acid contains SEQ ID NO: 31, 32, 33, or 34;
the sixth target nucleic acid contains SEQ ID NO: 41, 42, or 43;
the seventh target nucleic acid contains SEQ ID NO: 45, 46, or 47; and
the eighth target nucleic acid contains SEQ ID NO: 53, 55, 56, or 57.

14. The set of target nucleic acids of claim 13, wherein each target nucleic acid is 200-1,000 nucleotides in length.

15. A set of nucleic acids comprising one or more of
a first pair of primers, each containing an oligo-nucleotide selected from the human ADAR gene region;
a second pair of primers, each containing an oligo-nucleotide selected from the human CASP5 gene region;
a third pair of primers, each containing an oligo-nucleotide selected from the human FGF1 gene region;
a fourth pair of primers, each containing an oligo-nucleotide selected from the human ICSBP1 gene region;
a fifth pair of primers, each containing an oligo-nucleotide selected from the human IFI44 gene region;
a sixth pair of primers, each containing an oligo-nucleotide selected from the human PIK3CG gene region;
a seventh pair of primers, each containing an oligo-nucleotide selected from the human TAP2 gene region; and
an eighth pair of primers, each containing an oligo-nucleotide selected from the human TGFBRAP1 gene region.

16. The set of nucleic acids of claim 15, wherein each oligo-nucleotide is 14-50 nucleotides in length.

17. The set of nucleic acids of claim 16, wherein each oligo-nucleotide is 14-40 nucleotides in length.

18. The set of nucleic acids of claim 15, wherein
the oligo-nucleotides in the first pair of primers are, respectively, SEQ ID NOs: 75 and 76, SEQ ID NOs: 77 and 78, or SEQ ID NOs: 79 and 80;
the oligo-nucleotides in the second pair of primers are, respectively, SEQ ID NOs: 81 and 82, SEQ ID NOs: 81 and 177, SEQ ID NOs: 83 and 84, SEQ ID NOs: 83 and 178, SEQ ID NOs: 85 and 86, SEQ ID NOs: 87 and 88, SEQ ID NOs: 89 and 90, SEQ ID NOs: 89 and 181, SEQ ID NOs: 91 and 92, SEQ ID NOs: 93 and 94, SEQ ID NOs: 179 and 180, SEQ ID NOs: 182 and 183, or SEQ ID NOs: 184 and 185;
the oligo-nucleotides in the third pair of primers are, respectively, SEQ ID NOs: 95 and 96;
the oligo-nucleotides in the fourth pair of primers are, respectively, SEQ ID NOs: 97 and 98, SEQ ID NOs: 186 and 98, SEQ ID NOs: 99 and 100, SEQ ID NOs:99 and 187, SEQ ID NOs: 101 and 102, SEQ ID NOs: 101 and 188, SEQ ID NOs: 103 and 104, SEQ ID NOs: 103 and 189, SEQ ID NOs: 105 and 106, SEQ ID NOs: 105 and 190, SEQ ID NOs: 107 and 108, SEQ ID NOs: 191 and 108, SEQ ID NOs: 109 and 110, or SEQ ID NOs: 192 and 110;
the oligo-nucleotides in the fifth pair of primers are, respectively, SEQ ID NOs: 111 and 112, SEQ ID NOs: 113 and 114, SEQ ID NOs: 115 and 116, SEQ ID NOs:115 and 195, SEQ ID NOs: 117 and 118, SEQ ID NOs: 119 and 120, SEQ ID NOs: 121 and 122, SEQ ID NOs: 123 and 124, or SEQ ID NOs: 193 and 194;
the oligo-nucleotides in the sixth pair of primers are, respectively, SEQ ID NOs: 125 and 126, SEQ ID NOs: 125 and 196, SEQ ID NOs: 127 and 128, SEQ ID NOs: 197 and 128, SEQ ID NOs: 129 and 130, SEQ ID NOs: 129 and 198, SEQ ID NOs: 131 and 132, or SEQ ID NOs: 199 and 200;
the oligo-nucleotides in the seventh pair of primers are, respectively, SEQ ID NOs: 133 and 134, SEQ ID NOs: 201 and 134, SEQ ID NOs: 135 and 136, SEQ ID NOs: 202 and 136, SEQ ID NOs: 137 and 138, SEQ ID NOs: 203 and 138, SEQ ID NOs: 139 and 140, SEQ ID NOs: 204 and 140, SEQ ID NOs: 141 and 142, SEQ ID NOs: 205 and 142, SEQ ID NOs: 143 and 144, SEQ ID NOs: 206 and 144, SEQ ID NOs: 145 and 146, SEQ ID NOs: 145 and 207, SEQ ID NOs: 147 and 148, or SEQ ID NOs: 208 and 148; and
the oligo-nucleotides in the eighth pair of primers are, respectively, SEQ ID NOs: 149 and 150, SEQ ID NOs: 151 and 152, SEQ ID NOs: 153 and 154, SEQ ID NOs: 155 and 156, SEQ ID NOs: 157 and 158, SEQ ID NOs: 217 and 158, SEQ ID NOs: 159 and 160, SEQ ID NOs: 159 and 218, SEQ ID NOs: 161 and 162, SEQ ID NOs: 163 and 164, SEQ ID NOs:163 and 227, SEQ ID NOs: 165 and 166, SEQ ID NOs: 167 and 168, SEQ ID NOs: 169 and 170, SEQ ID NOs: 171 and 172, SEQ ID NOs: 173 and 174, SEQ ID NOs: 175 and 176, SEQ ID NOs: 209 and 210, SEQ ID NOs: 211 and 212, SEQ ID NOs: 213 and 214, SEQ ID NOs: 215 and 216, SEQ ID NOs: 219 and 220, SEQ ID NOs: 221 and 222, SEQ ID NOs: 223 and 224, SEQ ID NOs: 225 and 226, SEQ ID NOs: 228 and 229, or SEQ ID NOs: 230 and 176.

19. The set of nucleic acids of claim 18, wherein each oligo-nucleotide is 14-50 nucleotides in length.

20. The set of nucleic acids of claim 19, wherein each oligo-nucleotide is 14-40 nucleotides in length.

21. The set of nucleic acids of claim 18, wherein
the oligo-nucleotides in the second pair of primers are, respectively, SEQ ID NOs: 81 and 82, SEQ ID NOs: 81 and 177, SEQ ID NOs: 83 and 84, SEQ ID NOs: 83 and 178, SEQ ID NOs: 85 and 86, SEQ ID NOs: 87 and 88, or SEQ ID NOs: 179 and 180;
the oligo-nucleotides in the fourth pair of primers are, respectively, SEQ ID NOs: 97 and 98, SEQ ID NOs: 186 and 98, SEQ ID NOs: 99 and 100, SEQ ID NOs: 99 and 187, SEQ ID NOs: 101 and 102, SEQ ID NOs: 101 and 188, SEQ ID NOs: 103 and 104, SEQ ID NOs: 103 and 189, SEQ ID NOs: 105 and 106, or SEQ ID NOs: 105 and 190;
the oligo-nucleotides in the fifth pair of primers are, respectively, SEQ ID NOs: 111 and 112, SEQ ID NOs: 113 and 114, SEQ ID NOs: 115 and 116, SEQ ID NOs: 115 and 195, SEQ ID NOs: 117 and 118, or SEQ ID NOs: 193 and 194;
the oligo-nucleotides in the sixth pair of primers are, respectively, SEQ ID NOs: 125 and 126, SEQ ID NOs: 125 and 196, SEQ ID NOs: 127 and 128, SEQ ID NOs: 197 and 128, SEQ ID NOs: 129 and 130, or SEQ ID NOs: 129 and 198;
the oligo-nucleotides in the seventh pair of primers are, respectively, SEQ ID NOs: 133 and 134, SEQ ID NOs: 201 and 134, SEQ ID NOs: 135 and 136, SEQ ID NOs: 202 and 136, SEQ ID NOs: 137 and 138, or SEQ ID NOs: 203 and 138; and
the oligo-nucleotides in the eighth pair of primers are, respectively, SEQ ID NOs: 149 and 150, SEQ ID NOs: 151 and 152, SEQ ID NOs: 153 and 154, SEQ ID NOs: 209 and 210, SEQ ID NOs: 211 and 212, or SEQ ID NOs: 213 and 214.

22. A method of evaluating responsiveness of a subject to a drug, comprising
providing a nucleic acid sample from a subject;
determining a single nucleotide polymorphism genotype of a gene group that contains one or more of human ADAR, CASP5, FGF1, ICSBP1, IFI44, PIK3CG, TAP2, and TGFBRAP1 genes; and
comparing the single nucleotide polymorphism genotype with a predetermined single nucleotide polymorphism genotype,
whereby the subject is predicted to be responsive or non-responsive to the drug if the single nucleotide polymorphism genotype is identical to a predetermined single nucleotide polymorphism genotype.

23. The method of claim 22, wherein the subject has or is suspected of having a liver disorder.

24. The method of claim 23, wherein the subject has or is suspected of having hepatitis, liver fibrosis, or liver cirrhosis.

25. The method of claim 24, wherein the hepatitis is hepatitis C.

26. The method of claim 22, wherein the drug is type I interferon, ribavirin, or a combination thereof.

27. The method of claim 26, wherein the drug is interferon-alpha, interferon-beta, ribavirin, or a combination thereof.

28. The method of claim 22, wherein the nucleic acid sample contains one or more of
a first target nucleic acid obtained from amplification of the human ADAR gene nucleic acid template with a first pair of primers, each containing an oligo-nucleotide selected from the ADAR gene region;
a second target nucleic acid obtained from amplification of the human CASP5 gene nucleic acid template with a second pair of primers, each containing an oligo-nucleotide selected from the CASP5 gene region;
a third target nucleic acid obtained from amplification of the human FGF1 gene nucleic acid template with a third pair of primers, each containing an oligo-nucleotide selected from the FGF1 gene region;
a fourth target nucleic acid obtained from amplification of the human ICSBP1 gene nucleic acid template with a fourth pair of primers, each containing an oligo-nucleotide selected from the ICSBP1 gene region;
a fifth target nucleic acid obtained from amplification of the human IFI44 gene nucleic acid template with a fifth pair of primers, each containing an oligo-nucleotide selected from the IFI44 gene region;
a sixth target nucleic acid obtained from amplification of the human PIK3CG gene nucleic acid template with a sixth pair of primers, each containing an oligo-nucleotide selected from the PIK3CG gene region;
a seventh target nucleic acid obtained from amplification of the human TAP2 gene nucleic acid template with a seventh pair of primers, each containing an oligo-nucleotide selected from the TAP2 gene region; and
an eighth target nucleic acid obtained from amplification of the human TGFBRAP1 gene nucleic acid template with a eighth pair of primers, each containing an oligo-nucleotide selected from the TGFBRAP1 gene region;
wherein each target nucleic acid has a nucleotide at a single nucleotide polymorphism site and is 20-1,000 nucleotides in length.

29. The method of claim 28, wherein
the first target nucleic acid contains SEQ ID NO: 1, 2, 3, 4, 5, or 6;
the second target nucleic acid contains SEQ ID NO: 7, 8, 9, 10, 11, 12, or 13;
the third target nucleic acid contains SEQ ID NO: 14, 15, 16, 17, 18, 19, 20, 21, or 22;
the fourth target nucleic acid contains SEQ ID NO: 23, 24, 25, 26, 27, 28, 29, or 30;
the fifth target nucleic acid contains SEQ ID NO: 31, 32, 33, 34, 35, 36, 37, 38, 39, or 40;
the sixth target nucleic acid contains SEQ ID NO: 41, 42, 43, or 44;
the seventh target nucleic acid contains SEQ ID NO: 45, 46, 47, 48, 49, 50, 51, or 52; and
the first target nucleic acid contains SEQ ID NO: 53, 54, 55, 56, 57, 58, 59, 60, 61, 62, 63, 64, 65, 66, 67, 68, 69, 70, 71, 72, 73, or 74.

30. The method of claim 29, wherein
the first target nucleic acid contains SEQ ID NO: 1, 2, or 3;
the second target nucleic acid contains SEQ ID NO: 7, 8, 9, or 10;
the third target nucleic acid contains SEQ ID NO: 14 or 15;
the fourth target nucleic acid contains SEQ ID NO: 23, 24, 25, 26, 27, or 28;
the fifth target nucleic acid contains SEQ ID NO: 31, 32, 33, or 34;
the sixth target nucleic acid contains SEQ ID NO: 41, 42, or 43;
the seventh target nucleic acid contains SEQ ID NO: 45, 46, or 47; and
the eighth target nucleic acid contains SEQ ID NO: 53, 55, 56, or 57.

31. The method of claim 28, wherein
the oligo-nucleotides in the first pair of primers are, respectively, SEQ ID NOs: 75 and 76, SEQ ID NOs: 77 and 78, or SEQ ID NOs: 79 and 80;
the oligo-nucleotides in the second pair of primers are, respectively, SEQ ID NOs: 81 and 82, SEQ ID NOs: 81 and 177, SEQ ID NOs: 83 and 84, SEQ ID NOs: 83 and 178, SEQ ID NOs: 85 and 86, SEQ ID NOs: 87 and 88, SEQ ID NOs: 89 and 90, SEQ ID NOs: 89 and 181, SEQ ID NOs: 91 and 92, SEQ ID NOs: 93 and 94, SEQ ID NOs: 179 and 180, SEQ ID NOs: 182 and 183, or SEQ ID NOs: 184 and 185;
the oligo-nucleotides in the third pair of primers are, respectively, SEQ ID NOs: 95 and 96;
the oligo-nucleotides in the fourth pair of primers are, respectively, SEQ ID NOs: 97 and 98, SEQ ID NOs: 186 and 98, SEQ ID NOs: 99 and 100, SEQ ID NOs: 99 and 187, SEQ ID NOs: 101 and 102, SEQ ID NOs: 101 and 188, SEQ ID NOs: 103 and 104, SEQ ID NOs: 103 and 189, SEQ ID NOs: 105 and 106, SEQ ID NOs: 105 and 190, SEQ ID NOs: 107 and 108, SEQ ID NOs: 191 and 108, SEQ ID NOs: 109 and 110, or SEQ ID NOs: 192 and 110;
the oligo-nucleotides in the fifth pair of primers are, respectively, SEQ ID NOs: 111 and 112, SEQ ID NOs: 113 and 114, SEQ ID NOs: 115 and 116, SEQ ID NOs: 115 and 195, SEQ ID NOs: 117 and 118, SEQ ID NOs: 119 and 120, SEQ ID NOs: 121 and 122, SEQ ID NOs: 123 and 124, or SEQ ID NOs: 193 and 194;
the oligo-nucleotides in the sixth pair of primers are, respectively, SEQ ID NOs: 125 and 126, SEQ ID NOs: 125 and 196, SEQ ID NOs: 127 and 128, SEQ ID NOs: 197 and 128, SEQ ID NOs: 129 and 130, SEQ ID NOs: 129 and 198, SEQ ID NOs: 131 and 132, SEQ ID NOs: 199 and 200;
the oligo-nucleotides in the seventh pair of primers are, respectively, SEQ ID NOs: 133 and 134, SEQ ID NOs: 201 and 134, SEQ ID NOs: 135 and 136, SEQ ID NOs: 202 and 136, SEQ ID NOs: 137 and 138, SEQ ID NOs: 203 and 138, SEQ ID NOs: 139 and 140, SEQ ID NOs: 204 and 140, SEQ ID NOs: 141 and 142, SEQ ID NOs: 205 and 142, SEQ ID NOs: 143 and 144, SEQ ID NOs: 206 and 144, SEQ ID NOs: 145 and 146, SEQ ID NOs: 145 and 207, SEQ ID NOs: 147 and 148, SEQ ID NOs: 208 and 148; and
the oligo-nucleotides in the eighth pair of primers are, respectively, SEQ ID NOs: 149 and 150, SEQ ID NOs: 151 and 152, SEQ ID NOs: 153 and 154, SEQ ID NOs: 155 and 156, SEQ ID NOs: 157 and 158, SEQ ID NOs: 217 and 158, SEQ ID NOs: 159 and 160, SEQ ID NOs: 159 and 218, SEQ ID NOs: 161 and 162, SEQ ID NOs: 163 and 164, SEQ ID NOs:163 and 227, SEQ ID NOs: 165 and 166, SEQ ID NOs: 167 and 168, SEQ ID NOs: 169 and 170, SEQ ID NOs: 171 and 172, SEQ ID NOs: 173 and 174, SEQ ID NOs: 175 and 176, SEQ ID NOs: 209 and 210, SEQ ID NOs: 211 and 212, SEQ ID NOs: 213 and 214, SEQ ID NOs: 215 and 216, SEQ ID NOs: 219 and 220, SEQ ID NOs: 221 and 222, SEQ ID NOs: 223 and 224, SEQ ID NOs: 225 and 226, SEQ ID NOs: 228 and 229, or SEQ ID NOs: 230 and 176.

32. The method of claim 31, wherein
the oligo-nucleotides in the second pair of primers are, respectively, SEQ ID NOs: 81 and 82, SEQ ID NOs: 81 and 177, SEQ ID NOs: 83 and 84, SEQ ID NOs: 83 and 178, SEQ ID NOs: 85 and 86, SEQ ID NOs: 87 and 88, or SEQ ID NOs: 179 and 180;
the oligo-nucleotides in the fourth pair of primers are, respectively, SEQ ID NOs: 97 and 98, SEQ ID NOs: 186 and 98, SEQ ID NOs: 99 and 100, SEQ ID NOs: 99 and 187, SEQ ID NOs: 101 and 102, SEQ ID NOs: 101 and 188, SEQ ID NOs: 103 and 104, SEQ ID NOs: 103 and 189, SEQ ID NOs: 105 and 106, or SEQ ID NOs: 105 and 190;
the oligo-nucleotides in the fifth pair of primers are, respectively, SEQ ID NOs: 111 and 112, SEQ ID NOs: 113 and 114, SEQ ID NOs: 115 and 116, SEQ ID NOs: 115 and 195, SEQ ID NOs: 117 and 118, or SEQ ID NOs: 193 and 194;
the oligo-nucleotides in the sixth pair of primers are, respectively, SEQ ID NOs: 125 and 126, SEQ ID NOs: 125 and 196, SEQ ID NOs: 127 and 128, SEQ ID NOs: 197 and 128, SEQ ID NOs: 129 and 130, or SEQ ID NOs: 129 and 198;
the oligo-nucleotides in the seventh pair of primers are, respectively, SEQ ID NOs: 133 and 134, SEQ ID NOs: 201 and 134, SEQ ID NOs: 135 and 136, SEQ ID NOs: 202 and 136, SEQ ID NOs: 137 and 138, SEQ ID NOs: 203 and 138; and
the oligo-nucleotides in the eighth pair of primers are, respectively, SEQ ID NOs: 149 and 150, SEQ ID NOs: 151 and 152, SEQ ID NOs: 153 and 154, SEQ ID NOs: 209 and 210, SEQ ID NOs: 211 and 212, or SEQ ID NOs: 213 and 214.

33. The method of claim 22, wherein the predetermined single nucleotide polymorphism genotype is selected from a set of reference genotypes that include the genotypes listed in Table 2.
